(19) United States
(12) Patent Application Publication
(10) Pub. No.: US 2023/0279381 A1
(43) Pub. Date: Sep. 7, 2023

(54) METHODS AND COMPOSITIONS FOR PROFILING IMMUNE REPERTOIRE

(71) Applicant: 10x Genomics, Inc., Pleasanton, CA (US)

(72) Inventor: Wyatt James MCDONNELL, Pleasanton, CA (US)

(21) Appl. No.: 18/297,161

(22) Filed: Apr. 7, 2023

Related U.S. Application Data
(63) Continuation of application No. PCT/US2021/054307, filed on Oct. 8, 2021.
(60) Provisional application No. 63/090,148, filed on Oct. 9, 2020.

(51) Int. Cl. C12N 15/10

(52) U.S. Cl. CPC C12N 15/1037

(57) ABSTRACT

The present disclosure relates in some aspects to methods and compositions for analyzing a biomolecule, such as an antibody, and/or analyzing a repertoire comprising biomolecules, such as the repertoire of antibody binding specificities in a sample comprising one or more antibodies. Exemplary samples include serum and plasma, as well as monoclonal antibody compositions.

### FIELD

The present disclosure relates in some aspects to methods and compositions for analyzing a biomolecule, such as an antibody, and/or analyzing a repertoire comprising biomolecules, such as the repertoire of antibody binding specificities in a subject.

### BACKGROUND

While there are forward approaches capable of isolating antibodies with specificity to or binding capacity to a small number of antigens, there are few approaches that can efficiently screen the total antigen binding specificity and/or binding capacity of an antibody repertoire, such as the repertoire of secreted antibodies produced by plasma cells. Approaches such as protein microarrays have been used to screen for antibody specificities in serum samples. However, these array-based approaches may distort and/or mask the antigens or epitopes recognized by antibodies in solution and/or in vivo. There are needs for improved methods for analyzing biomolecules and repertoires thereof, including the repertoire of antibody binding specificities in a sample. The present disclosure addresses these and other needs.

### BRIEF SUMMARY

In some aspects, provided herein is a method for processing and/or analyzing a sample comprising one or more antibodies or antigen-binding fragments thereof, such as a serum or plasma sample. In some embodiments, the method comprises contacting the sample with a population (e.g., library) of engineered cells, such as yeast cells. In some embodiments, the population comprises a cell engineered to express an antigen or epitope, and expression of the antigen or epitope is heterologous to the cell.

In some embodiments, the engineered cell comprises (e.g., in the engineered genome) a nucleic acid sequence corresponding to the antigen or epitope. In some embodiments, the nucleic acid sequence uniquely corresponds to the antigen or epitope. In some embodiments, the nucleic acid sequence or a complement thereof is present in the engineered genome of the cell, e.g., in an expression unit introduced into the cell’s genome, for example, at a constitutively expressed site. In some embodiments, the nucleic acid sequence or a complement thereof is present in a transcript of an expression unit introduced into the cell’s genome. In some embodiments, the nucleic acid sequence or a complement thereof is attached to the antigen or epitope expressed by the cell, e.g., through chemical or enzymatic conjugation. In some embodiments, a sequence of the nucleic acid sequence or a complement thereof encodes all or a portion of the antigen or epitope. In some embodiments, the nucleic acid sequence is a barcode that corresponds to the antigen or epitope, and the barcode or a complement thereof does not comprise a sequence encoding the antigen or epitope.

In some aspects, all or a subset of the engineered cells of the population are bound by one or more antibodies or antigen-binding fragments thereof, e.g., through specific binding between an antibody or antigen binding fragment thereof and an antigen or epitope expressed by a cell of the population. In some embodiments, the method comprises selecting or enriching engineered cells expressing antigens or epitopes specifically bound by the one or more antibodies or antigen-binding fragments thereof.

In some embodiments, the cells bound by an antibody or antigen-binding fragment thereof are partitioned, wherein upon partitioning, a partition comprises an engineered cell bound by an antibody or antigen-binding fragment thereof and a partition barcode sequence in the partition. In some embodiments, the partition comprises a gel bead comprising a plurality of molecules which include the partition barcode sequence. In some embodiments, a first barcoded nucleic acid molecule is generated in the partition, wherein the first barcoded nucleic acid molecule comprises (i) a sequence of the nucleic acid sequence corresponding to the antigen or epitope, or a complement thereof, and (ii) a sequence of the partition barcode sequence, or a complement thereof.

In some aspects, the sample comprises one or more monoclonal antibodies each comprising an antibody barcode sequence. In some embodiments, the antibody barcode sequence corresponds to the monoclonal antibody that it is attached to. In some embodiments, in addition to the first barcoded nucleic acid molecule, a second barcoded nucleic acid molecule is generated in the partition, wherein the second barcoded nucleic acid molecule comprises (i) a sequence of the antibody barcode sequence, or a complement thereof, and (ii) a sequence of the partition barcode sequence, or a complement thereof. The first and second barcoded nucleic acid molecules generated in each single cell partition may be detected or otherwise analyzed, e.g., by sequencing, to reveal the identity of the antigen or epitope and the identity of the monoclonal antibody bound to the cell, respectively, thereby providing a method for epitope mapping of the antibody. In some embodiments, epitope mapping of the one or more monoclonal antibodies are performed in parallel and/or in a highly multiplexed manner.

In some aspects, provided herein is a method of analyzing a sample, comprising: contacting a sample with a population of cells, wherein the sample comprises a plurality of antibody molecules or antigen-binding fragments thereof, wherein the population comprises: (i) a first cell that is engineered or otherwise modified to express a first antigen or epitope and that comprises a nucleic acid molecule comprising a first nucleic acid sequence corresponding to the first antigen or epitope, and (ii) a second cell that is engineered or otherwise modified to express a second antigen or epitope and that comprises a nucleic acid molecule comprising a second nucleic acid sequence corresponding to the second antigen or epitope, wherein the first antigen or epitope of the first cell is bound by one or more antibody molecules or antigen-binding fragments in the sample while the second antigen or epitope of the second cell is not, wherein the first cell with the bound one or more antibody molecules or antigen-binding fragments is partitioned in a partition, the partition comprising a plurality of barcode nucleic acid molecules which comprise a plurality of partition barcode sequences (e.g., which may be or include a common partition-specific barcode sequence), and wherein a barcoded nucleic acid molecule is generated in the partition, and the barcoded nucleic acid molecule comprises (i) the first nucleic acid sequence or a complement thereof and (ii) a partition barcode sequence (e.g., the common partition-specific barcode sequence) or a complement thereof. In some embodiments, the sample can comprise a bodily fluid. In some embodiments, the sample can be a blood sample, a serum sample, or a plasma sample.

In any of the embodiments herein, the plurality of antibody molecules or antigen-binding fragments thereof, may comprise two or more antibody molecules or antigen-binding fragments thereof having different antigen-binding specificities and/or affinities. In any of the embodiments herein, the plurality of antibody molecules or antigen-binding fragments thereof, may comprise two or more antibody molecules or antigen-binding fragments thereof having the same antigen-binding specificities and/or affinities. In any of the embodiments herein, the sample plurality of antibody molecules or antigen-binding fragments thereof, may comprise an antibody molecule or antigen-binding fragment thereof that recognizes two or more different antigens or epitopes.

In any of the embodiments herein, the plurality of antibody molecules or antigen-binding fragments thereof, may comprise one or more monoclonal antibodies. In some embodiments, the one or more monoclonal antibodies may be coupled to one or more antibody barcode sequences.

In any of the embodiments herein, the first and/or second antigens or epitopes can be heterologous to the first and second cells, respectively. In some embodiments, the first and second cells can be yeast cells, and the first and/or second antigens or epitopes are of viral, fungal, bacterial, plant, and/or mammalian (e.g., human) origin.

In any of the embodiments herein, the first and/or second antigens or epitopes can be peptides or proteins. In any of the embodiments herein, the first and/or second antigens or epitopes may be intracellular, extracellular, transmembrane, cell surface, and/or secreted antigens or epitopes. In some embodiments, the secreted antigens or epitopes can be captured on the cell surface by a capture agent or by a matrix (e.g., matrix of a cell bead) encapsulating the cell.

In any of the embodiments herein, the first and second nucleic acid sequences may be different, thereby identifying the first cell and the second cell as expressing different antigens or epitopes. In any of the embodiments herein, the first and second nucleic acid sequences can be directly or indirectly coupled to the first and second antigens or epitopes, respectively. In some embodiments, the coupling can be through chemical conjugation and/or enzymatic conjugation. In some embodiments, the enzymatic conjugation can be a sortase-catalyzed cell surface conjugation.

In any of the embodiments herein, the first and second cells can be engineered by introducing into the cells an expression system capable of expressing the first and second antigens or epitopes, respectively, in the cells. In some embodiments, the expression system capable of introducing expressing the first and second antigen into the first and second cells, respectively, can be introduced into the genome of the first and second cells. In some embodiments, the expression system can comprise coding sequences for the first and second antigens or epitopes and optionally affinity tags (e.g., His-tag) to be fused to the first and second antigens or epitopes. In some embodiments, the coding sequences can comprise or form part of the nucleic acid sequences corresponding to the first and second antigens or epitopes. In some embodiments, the first and second cells constitutively express the first and second antigens or epitopes, respectively, from the expression system. In some embodiments, the expression system can comprise a first and second antigen barcode sequence or a complement thereof corresponding to the first and second antigens or epitopes, respectively, wherein the first and second antigen barcode sequences or complements thereof are distinct from coding sequences for the first and second antigens or epitopes, respectively. In some embodiments, the expression system can comprise a sequence or a complement thereof configured to couple to a barcode nucleic acid molecule of the plurality of barcode nucleic acid molecules in the partition. In some embodiments, a transcript of the expression system can be coupled to a barcode nucleic acid molecule of the plurality of barcode nucleic acid molecules in the partition. In some embodiments, the transcript can comprise a coding sequence for the first or second antigen or epitope, an antigen barcode sequence or a complement thereof corresponding to the first or second antigen or epitope, and a sequence or a complement thereof configured to couple the transcript to a barcode nucleic acid molecule of the plurality of barcode nucleic acid molecules in the partition.

In any of the embodiments herein, wherein during or after the contacting, cells of the population of cells bound by one or more of the plurality of antibody molecules or antigen-binding fragments thereof in the sample can be enriched, purified, isolated, sorted, and/or separated (e.g., from cells of the population of cells not bound by one or more of the plurality of antibody molecules or antigen-binding fragments thereof in the sample), optionally wherein cells of the population of cells not bound by one or more of the plurality of antibody molecules or antigen-binding fragments thereof in the sample can be enriched, purified, isolated, sorted, and/or separated (e.g., from cells of the population of cells bound by one or more of the plurality of antibody molecules or antigen-binding fragments thereof in the sample). In some embodiments, the cells of the population of cells bound by one or more of the plurality of antibody molecules or antigen-binding fragments thereof in the sample can be sorted or purified using a cytometer. In some embodiments, the cells of the population of cells bound by one or more of the plurality of antibody molecules or antigen-binding fragments thereof can be sorted or purified using fluorescence-activated cell sorting (FACS), e.g., using a fluorescently labeled antibody (e.g., an anti-Fc antibody) that recognizes the antibody molecules or antigen-binding fragments thereof bound on the one or more cells. In some embodiments, the cells of the population of cells bound by the one or more of the plurality of antibody molecules or antigen-binding fragments thereof can be sorted or purified using magnetic-activated cell sorting (MACS), e.g., using an antibody (e.g., an anti-Fc antibody coupled to a magnetic bead) that recognizes the antibody molecules or antigen-binding fragments thereof bound on the one or more cells. In some embodiments, the cells of the plurality of cells bound by the one or more of the plurality of antibody molecules or antigen-binding fragments thereof can be sorted or purified using buoyancy-activated cell sorting (BACS), e.g., using an antibody (e.g., an anti-Fc antibody coupled to a microbubble) that recognizes the one or more of the plurality of antibody molecules or antigen-binding fragments thereof bound on the cells of the population of cells.

In any of the embodiments herein, one or more of the barcoded nucleic acid molecules are is detected and/or analyzed by nucleic acid sequencing.

In any of the embodiments herein, the method may further comprise optically imaging one or more of the: one or more of the plurality of antibody molecules or antigen-binding fragments thereof, antibody barcode sequence, antigen or epitope, antigen barcode sequence, cell, partition, partition barcode sequence, and barcoded nucleic acid molecule.

In any of the embodiments herein, the method may not comprise optically imaging one or more of the: one or more of the plurality of antibody molecules or antigen-binding fragments thereof, antibody barcode sequence, antigen or epitope, antigen barcode sequence, cell, partition, partition barcode sequence, and barcoded nucleic acid molecule.

In some aspects, provided herein is a method for analyzing a sample, comprising: contacting a sample with a population of cells, wherein the sample comprises a monoclonal antibody or antigen-binding fragment thereof coupled to an antibody barcode sequence, wherein the population comprises a cell that is engineered or otherwise modified to express an antigen or epitope and that comprises an antigen barcode sequence corresponding to the antigen or epitope, wherein the antigen or epitope expressed by the cell is bound by the monoclonal antibody or antigen-binding fragment thereof, wherein the cell with the bound monoclonal antibody or antigen-binding fragment thereof is partitioned in a partition, the partition comprising a plurality of barcode nucleic acid molecules which comprise a plurality of partition barcode sequences (e.g. which may be or include a common partition-specific barcode sequence), and wherein a first barcoded nucleic acid molecule and a second barcoded nucleic acid molecule are generated in the partition, the first barcoded nucleic acid molecule comprises (i) the antibody barcode sequence or a complement thereof and (ii) a partition barcode sequence (e.g., the common partition-specific barcode sequence) or a complement thereof, and the second barcoded nucleic acid molecule comprises (i) the antigen barcode sequence or a complement thereof and (ii) a partition barcode sequence (e.g., the common partition-specific barcode sequence) or a complement thereof. In some embodiments, the sample may comprise a plurality of monoclonal antibodies or antigen-binding fragments thereof. In some embodiments, the plurality of monoclonal antibodies or antigen binding fragments thereof comprises two or more monoclonal antibodies or antigen-binding fragments thereof having different antigen-binding specificities and/or affinities. In some embodiments, the plurality of monoclonal antibodies or antigen binding fragments thereof may comprise a monoclonal antibody or antigen-binding fragment thereof that recognizes two or more different antigens or epitopes. In some embodiments, the population may comprise cells engineered or otherwise modified to express a panel of candidate epitopes, comprising the antigen or epitope, for the monoclonal antibody or antigen-binding fragment thereof. In some embodiments, wherein during or after the contacting, cells of the population of cells bound by monoclonal antibodies or antigen-binding fragments thereof in the sample can be enriched, purified, isolated, sorted, and/or separated (e.g., from cells of the population of cells not bound by monoclonal antibodies or antigen-binding fragments thereof in the sample), optionally wherein cells of the population of cells not bound by monoclonal antibodies or antigen-binding fragments thereof in the sample can be enriched, purified, isolated, sorted, and/or separated (e.g., from cells of the population of cells bound by monoclonal antibodies or antigen-binding fragments thereof in the sample). In some embodiments, the population of cells may be yeast cells.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an exemplary microfluidic channel structure for partitioning individual biological particles.

FIG. 2 shows an exemplary microfluidic channel structure for the controlled partitioning of beads into discrete droplets.

FIG. 3 shows an exemplary barcode carrying bead.

FIG. 4 illustrates another example of a barcode carrying bead.

FIG. 5 schematically illustrates an exemplary microwell array.

FIG. 6 schematically illustrates an example workflow for processing nucleic acid molecules.

FIG. 7 shows an example of a barcoded bead that may be used in a partition such as a droplet to couple a barcode (e.g., a partition-specific barcode) and one or more analytes (e.g., an antibody bound to an antigen or epitope in a cell, mRNAs, etc.) of a single cell, thereby associating said one or more analytes with the single cell.

FIG. 8 shows an illustration of the conversion of barcoded analytes into sequencing libraries.

FIG. 9 shows an example of simultaneous measurement of secreted analysts, mRNAs, cell surface proteins, paired αβ T-cell receptor sequences, and antigen binding specificity.

FIG. 10 schematically illustrates examples of labelling agents.

FIG. 11 depicts an example of a barcode carrying bead.

FIGS. 12 A-C schematically depict an example workflow for processing nucleic acid molecules.

FIG. 13 shows a computer system that is programmed or otherwise configured to implement methods provided herein.

FIG. 14 shows exemplary uses of sortase labeling to label an antibody with an oligonucleotide barcode. The antigen can be fused to a sortase, and the antibody of interest can be tagged with a sortase recognition sequence (LPXTG) or a sortase acceptor peptide (e.g., an oligoglycine, (G)ₙ). The antibody and cell population expressing the sortase-fused antigen can be incubated together in the presence of an oligonucleotide-barcode fused to a sortase recognition sequence (LPXTG) or a sortase acceptor peptide (e.g., an oligoglycine, (G)ₙ). Binding of the antibody to the antigen will result in labeling of the antibody with the oligonucleotide barcode. In some cases, the antibody can be a secreted antibody engineered in to the cell line, and binding of the secreted antibody to the antigen can be detected based on labeling of the antibody with the oligonucleotide barcode.

### DETAILED DESCRIPTION

The practice of the techniques described herein may employ, unless otherwise indicated, conventional techniques and descriptions of organic chemistry, polymer technology, molecular biology (comprising recombinant techniques), cell biology, biochemistry, and sequencing technology, which are within the skill of those who practice in the art. Such conventional techniques comprise polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W. H. Freeman Pub., New York, N.Y.; and Berg et al. (2002) Biochemistry, 5^{th} Ed., W. H. Freeman Pub., New York, N.Y., all of which are herein incorporated in their entirety by reference for all purposes.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In one aspect, provided herein is a method for analyzing a sample, comprising contacting the sample, e.g., a serum or plasma sample, with a population of engineered cells, e.g., yeast cells each expressing a single antigen or epitope of human, viral, bacterial, or other origin. In some embodiments, the sample comprises a plurality of antigen binding molecules, e.g., antibody molecules or antigen-binding fragments thereof. In some embodiments, the sample comprises a plurality of molecules of the same antibody (e.g., antibodies that belong to the same clone or having the same heavy chain and/or light chain CDR sequences), and/or two or more molecules of different antibodies (e.g., antibodies of different clones or having one or more different heavy chain and/or light chain CDR sequences).

In some embodiments, the cell population, e.g., population of engineered cells referenced above, comprises a cell that is engineered to express an antigen or epitope and that comprises a nucleic acid sequence corresponding to the antigen or epitope. In some embodiments, the nucleic acid sequence comprises a sequence (e.g., a barcode sequence) that identifies the corresponding antigen or epitope.

In some embodiments, a cell-antibody complex is formed upon binding of the antigen or epitope expressed by the cell to one or more antibody molecules or antigen-binding fragments in the sample. In some embodiments, the cell-antibody complex is selected, e.g., through cell sorting or a separation method, and is partitioned in a partition. In some embodiments, the cell-antibody complex is separated from one or more cells of the population that are not bound by an antibody molecule or antigen-binding fragment in the sample. In some embodiments, the cell-antibody complex is selectively partitioned in a partition while a cell not bound by an antibody molecule or antigen-binding fragment in the sample is not partitioned. In some embodiments, the partition comprising the cell-antibody complex is distinguishable, and may be separated, from a partition that comprises a cell not bound by an antibody molecule or antigen-binding fragment in the sample.

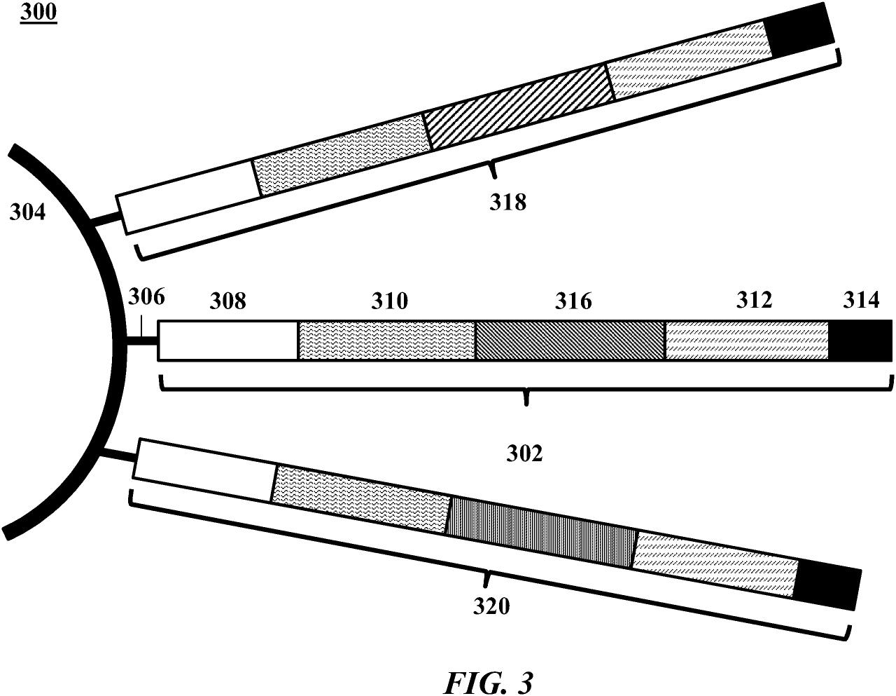

In some embodiments, the partition comprises a plurality of barcode nucleic acid molecules which comprise a plurality of partition barcode sequences, in which the plurality of partition barcode sequences may be or include a common partition-specific barcode sequence as described, for example in the disclosures related to FIG. 3, in particular in reference to 310. In some embodiments, a barcoded nucleic acid molecule is generated in the partition, and the barcoded nucleic acid molecule comprises (i) the nucleic acid sequence or a complement thereof that corresponds to the antigen or epitope expressed by the cell, and (ii) a partition barcode sequence, or the partition-specific barcode sequence or a complement thereof.

In some embodiments, the cell is a first cell and the antigen or epitope is a first antigen or epitope, and the population further comprises a second cell that is engineered to express a second antigen or epitope and that comprises a second nucleic acid sequence corresponding to the second antigen or epitope. In some embodiments, the second nucleic acid sequence comprises a sequence (e.g., a barcode sequence) that identifies the corresponding antigen or epitope. In some embodiments, the first and second barcode sequences identify the first and second antigens or epitopes, respectively. In some embodiments, the second antigen or epitope of the second cell is not bound by an antibody molecule or antigen-binding fragment in the sample. In some embodiments, the second cell does not form a cell-antibody complex, and the second cell (or a partition comprising the second cell) is distinguishable, and may be separated, from the first cell-antibody complex (or a partition comprising the cell-antibody complex). In some embodiments, a barcoded nucleic acid molecule comprising (i) the second nucleic acid sequence or a complement thereof and (ii) a partition barcode sequence or the partition-specific barcode sequence or a complement thereof is not generated in a method disclosed herein, thereby identifying the sample as containing one or more antibody molecules recognizing the first antigen or epitope but not containing one or more antibody molecules recognizing the second antigen or epitope. In some embodiments, a method disclosed herein is used to profile the antigen binding capacities, including antigen binding specificities, of the antigen binding molecules in the sample. In some embodiments, a method disclosed herein is used to analyze one or more monoclonal antibodies, for example, for epitope mapping, by expressing a panel of candidate epitopes in the population of cells.

In some embodiments, provided herein are methods comprising contacting a library of cells (e.g., a library of engineered eukaryotic cells such as a yeast display library) with a biological sample, such as a sample containing one or more antibodies (e.g., a serum or plasma sample). In some embodiments, the method further comprises selecting and/or separating out cells of the library that are bound by one or more components, e.g., antibodies, in the biological sample. For example, clones, e.g., engineered cells, coated by an antibody in a cell library can be selected and analyzed (e.g., by sequencing) to determine interaction between the cells and the one or more components in the biological sample, such as a reactivity between an antigen or epitope (e.g., derived from human extracellular proteins) expressed by one or more engineered cells, e.g, yeast clones, and one or more antibodies in the sample. In some embodiments, the reactivity can be confirmed by another assay, such as ELISA. In some embodiments, a method disclosed herein is used to select candidates for an assay, for example, antigens for LIBRA-seq (linking B cell receptor to antigen specificity through sequencing) can be selected using the methods disclosed herein.

In some embodiments, provided herein are methods comprising engineering a pool of eukaryotic cells such as yeast cells. In some embodiments, a library of cells are produced, and each cell: 1) uniquely displays a human, viral, bacterial, or other antigen of interest, and 2) stably transcribes the antigen from its having been cloned into a constitutively expressed site with a capture sequence that can be captured in each cell and a barcode that is unique to the antigen.

As the antigen is displayed on the surface of the yeast cell, each cell can putatively be used to study the antigen specificity found in the plasma of each individual. By combining serum and the yeast cell library, some cells will be selectively coated with antibody, and cells expressing antigens not recognized by antibodies in the serum will not be coated with antibody. Using magnetic, buoyant, or other selection methods targeting the Fc region of the bound antibodies, yeast cells, expressing antigens bound by the serum antibodies can be selectively enriched for capture in an emulsion droplet based system such as the 10x Genomics Chromium™ system, such that an antibody-bound cell is encapsulated per droplet. This enables the capture of yeast cells, expressing antigens of interest, and selective targeted amplification of the antigen barcode encoded by each cell. The results can be read out with short-read sequencing.

In one additional embodiment, for samples containing a known antibody molecule or antigen-binding fragment thereof, an anti-idiotypic antibody may be used to selectively enrich for cells expressing antigens bound by the serum antibodies. For instance, an antibody specific for the idiotope (e.g., epitopes in the hypervariable complementarity determining region(s) (CDRs)) of the known antibody molecule or antigen-binding fragment thereof may be used for selective enrichment.

In some embodiments, an incubation or culture period is used after initial selection to increase the frequency of antigen-expressing clones. In some embodiments, the cells are incubated or cultured to generate a secondary culture that could be re-screened/re-selected with additional serum or plasma. In some embodiments, cellular libraries containing multiple variants of the same antigen (e.g., 80 variants of the same protein) are used to assess specificity of the serum/plasma profile. In some embodiments, antigen sequences could be modified to contain a tag (e.g., a hexa-histidine or other protein tag) to facilitate purification and rapid oligonucleotide conjugation to make a reporter barcoded antigen (e.g., an antigen having a reporter barcode oligonucleotide comprising a reporter nucleic acid sequence) for isolation of specific antibodies in a separate workflow. In some embodiments, cell libraries are used to perform epitope mapping by incubation with one or more reporter barcoded monoclonal antibodies, where detection of the antigen barcode in the cell, e.g., yeast cell, and the reporter barcoded antibody of interest indicates binding. Epitope variant panels would allow for fine mapping of the antigen specificity of a given antibody. In some embodiments, a method disclosed herein comprises a visual detection format by expression of a fluorophore or other detection molecule and sorting fluorescent droplets on the basis of the presence (or absence) of the cells, e.g., yeast cells, and/or the presence (or absence) of a fluorescent or magnetic tag on antibody/antibodies of interest.

Advantages of the certain embodiments herein include, first, by utilizing engineered eukaryotic cells (e.g., yeast cells), the expressed antigens can be appropriately translationally, co-translationally, or post-translationally modified in a eukaryotic setting - a complex bioprocess that generally cannot be reliably reproduced in vitro. Secondly, by using a cellular library, proper epitope display and conformational preservation is ensured, allowing the identification of antibody responses against linear, non-linear, and complex epitopes, unlike microarrays. Last but not least, cells expressing an identified antigen of interest could be used as source material for cell line engineering and bioproduction of a given antigen, enabling the efficient capture of antibodies with a given specificity using reporter barcoded antigens and single cell genomic technologies.

### II. Binding Molecules and Binding Partners

In some aspects, provided herein are methods and compositions that enable the analysis of a binding interaction between a binding molecule (e.g., an antibody in a serum or plasma sample, or a monoclonal antibody such as a barcoded monoclonal antibody) and a binding partner (e.g., an antigen or epitope displayed by a cell). In some embodiments, a method disclosed herein comprises contacting a binding molecule with a cell population comprising one or more cells comprising a binding partner (e.g., an antigen or epitope) and a nucleic acid molecule comprising a nucleic acid sequence corresponding to the binding partner, to allow binding between molecules of the binding molecule and the binding partner of the one or more cells.

In some embodiments, at least a portion or all of the binding partner (e.g., an antigen or epitope) is present or expressed on a surface of a cell. In some embodiments, at least a portion or all of the binding partner (e.g., an antigen or epitope) is extracellular and is directly or indirectly coupled to a cell. In some embodiments, the binding partner (e.g., an antigen or epitope) is present or expressed in a cell. In some embodiments, the binding partner (e.g., an antigen or epitope) is secreted or soluble, and is configured to directly or indirectly coupled to a cell, e.g., by a cell-surface capture agent (e.g., a molecule recognizing a cell surface molecule and the binding partner) and/or by a matrix surrounding the cell (e.g., the matrix of a cell bead). See, e.g., U.S. Pat. 10,428,326 and U.S. Pat. 10,590,244, which are incorporated by reference in their entirety, for exemplary cell bead generation systems and methods.

In some embodiments, all or a subset of the cells of the population are partitioned into a plurality of partitions, wherein upon partitioning, a partition of the plurality of partitions comprises a cell of the population or subset thereof and a nucleic acid barcode sequence in the partition. In some embodiments, the cell in the partition is bound to one or more molecules of the binding molecule.

In some embodiments, when the binding molecule binds the binding partner, a barcoded nucleic acid molecule is generated in the partition, wherein the barcoded nucleic acid molecule comprises (i) a nucleic acid sequence corresponding to the binding partner and (ii) a sequence corresponding to the nucleic acid barcode sequence in the partition. The barcoded nucleic acid molecule may be analyzed to analyze the binding between the binding molecule and the binding partner.

In some aspects, provided herein is a method of analyzing the repertoire of antigen binding specificities of antibodies in a sample, comprising: contacting a sample comprising an antibody or antigen-binding fragment thereof with a cell population comprising one or more cells expressing an antigen or epitope on the cell surface and comprising a first nucleic acid barcode sequence corresponding to the antigen or epitope, wherein all or a subset of the cells of the population are partitioned into a plurality of partitions, wherein upon partitioning, a partition of the plurality of partitions comprises a cell of the population or subset thereof and a second nucleic acid barcode sequence in the partition, wherein when the antibody or antigen-binding fragment thereof binds the antigen or epitope expressed on the cells, a barcoded nucleic acid molecule is generated in the partition, wherein the barcoded nucleic acid molecule comprises (i) a sequence corresponding to the first nucleic acid barcode sequence and (ii) a sequence corresponding to the second nucleic acid barcode sequence, and wherein the barcoded nucleic acid molecule is analyzed to analyze the binding between the antibody or antigen-binding fragment thereof and the antigen or epitope.

In some aspects, a method provided herein further comprises analyzing an expression or presence status of the antigen or epitope of the cell in a partition of the plurality of partitions. In some aspects, the cell population further comprises one or more cells that do not express the antigen or epitope, one or more cells engineered to express or to increase expression of the antigen or epitope, and/or one or more cells engineered to not express or to decrease expression of the antigen or epitope. In some embodiments, the plurality of partitions comprise a partition comprising a cell engineered or otherwise modified to express or to increase expression of the antigen or epitope. In some embodiments, the plurality of partitions comprise a partition comprising a cell engineered or otherwise modified to not express or to decrease expression of the antigen or epitope. In some embodiments, the plurality of partitions comprise a partition comprising a cell that does not express the antigen or epitope and that comprises a modification to eliminate or decrease expression of the antigen or epitope.

### A. Antigens and Antigen Binding Molecules

In some embodiments, an antigen binding molecule herein may include any molecule that specifically binds an antigenic determinant. Examples of antigen binding molecules are immunoglobulins and derivatives, e.g. fragments, thereof, and generally refer to a portion of a complete antibody (e.g., comprising each domain of the light and heavy chains respectively) capable of binding the same epitope/antigen as the complete antibody, albeit not necessarily to the same extent. Although multiple types of antigen binding molecules are possible, an antigen binding fragment typically comprises at least one pair of heavy and light chain variable regions (VH and VL, respectively) held together (e.g., by disulfide bonds) to preserve the antigen binding site and does not contain all or a portion of the Fc region. Antigen binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an antigen binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide. In some embodiments, the antibody can be a monoclonal antibody (mAb), a recombinant bispecific monoclonal antibody such as a Bi-specific T-cell engager (BiTE), a simultaneous multiple interaction T-cell engaging (SMITE) bispecific antibody, or a bi-, tri-, or tetra-valent antibody.

In some embodiments, an antigen binding molecule herein may include an immunoglobulin molecule, e.g., a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or µ (IgM), some of which may be further divided into subtypes, e.g. γ₁ (IgG₁), γ₂ (IgG₂), γ₃ (IgG₃), γ₄ (IgG₄), α₁ (IgAi) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

In some embodiments, an antigen binding molecule herein may include a BCR, which is composed of two genes IgH and IgK (or IgL) coding for antibody heavy and light chains. Immunoglobulins are formed by recombination among gene segments, sequence diversification at the junctions of these segments, and point mutations throughout the gene. Each heavy chain gene contains multiple copies of three different gene segments—a variable ‘V’ gene segment, a diversity ‘D’ gene segment, and a joining ‘J’ gene segment. Each light chain gene contains multiple copies of two different gene segments for the variable region of the protein—a variable ‘V’ gene segment and a joining ‘J’ gene segment. The recombination can generate a molecule with one of each of the V, D, and J segments. Furthermore, several bases may be deleted and others added (called N and P nucleotides) at each of the two junctions, thereby generating further diversity. After B cell activation, a process of affinity maturation through somatic hypermutation occurs. In this process progeny cells of the activated B cells accumulate distinct somatic mutations throughout the gene with higher mutation concentration in the CDR regions leading to the generation of antibodies with higher affinity to the antigens. In addition to somatic hypermutation activated B cells undergo the process of isotype switching. Antibodies with the same variable segments can have different forms (isotypes) depending on the constant segment. Whereas all naive B cells express IgM (or IgD), activated B cells mostly express IgG but also IgM, IgA and IgE. This expression switching from IgM (and/or IgD) to IgG, IgA, or IgE occurs through a recombination event causing one cell to specialize in producing a specific isotype. A unique nucleotide sequence that arises during the gene arrangement process can similarly be referred to as a clonotype.

The antigens may include any antigen against which it is desired to test binding of an antigen binding molecule herein. In some embodiments, antigens are derived from bacteria, fungi, viruses, or allergens. In some embodiments, antigens are derived from internal sources, such as tumor cells or self-proteins (e.g. self-antigens). In some embodiments, the tumor antigen is in a tumor lysate. Self-antigens are antigens present on an organism’s own cells. Self-antigens do not normally stimulate an immune response, but may in the context of autoimmune diseases, such as Type I Diabetes or Rheumatoid Arthritis, Multiple Sclerosis (and other demyelinating disorders). In some embodiments, the antigen is a neoantigen. Neoantigens are antigens that are absent from the normal human genome, but are created within oncogenic cells as a result of tumor-specific DNA modifications that result in the formation of novel protein sequences. Exemplary viral antigens include HIV antigens, Ebola antigen, HPV antigens, and EBV antigens, which are purified or delivered as a mixture, or delivered as killed or attenuated virus or virus fragments. In some embodiments, the HPV antigens are derived from the oncogenes E6 and E7 of HPV 16. In some embodiments, the antigen is a non-protein antigen, such as a lipid, glycolipid, or polysaccharide.

For example, the antigens can include, but are not limited to, any antigens associated with a pathogen, including viral antigens, fungal antigens, bacterial antigens, helminth antigens, parasitic antigens, ectoparasite antigens, protozoan antigens, or antigens from any other infectious agent. Antigens can also include any antigens associated with a particular disease or condition, whether from pathogenic or cellular sources, including, but not limited to, cancer antigens, antigens associated with an autoimmune disease (e.g., diabetes antigens), allergy antigens (allergens), mammalian cell molecules harboring one or more mutated amino acids, proteins normally expressed pre- or neo-natally by mammalian cells, proteins whose expression is induced by insertion of an epidemiologic agent (e.g., virus), proteins whose expression is induced by gene translocation, and proteins whose expression is induced by mutation of regulatory sequences. These antigens can be native antigens or genetically engineered antigens which have been modified in some manner (e.g., sequence change or generation of a fusion protein). It will be appreciated that in some embodiments (e.g., when the antigen is expressed by the yeast vehicle from a recombinant nucleic acid molecule), the antigen can be a protein or any epitope of immunogenic domain thereof, a fusion protein, or a chimeric protein, rather than an entire cell or microorganism.

In some embodiments, the antigen is a carbohydrate antigen. For example, the epitope can be a cell-surface oligosaccharides (e.g., an oligosaccharide linked to a lipid or protein on the cell surface). In some embodiments, the carbohydrate antigen is a tumor antigen, such as the Lewis x and y antigens, their sialylated counterparts, or a gangliosides. Gangliosides are glycosphingolipids, which are ceramide-linked oligosaccharides with at least one terminal sialic acid residue.

Antigen or antibody specificity is a measure of an antibody’s ability to bind uniquely to a specific antigen. In some cases, a particular epitope recognized by an antigen might appear on more than one protein antigen (cross-reactivity or low specificity). An antibody with high specificity would result in less cross-reactivity with different antigens. The specificity of an antibody for a target antigen can be assessed according to the methods provided herein by comparing antibody binding in cell populations expressing the target antigen with antibody binding to cell populations that do not express the target antigen. In comparison with specificity, affinity of an antibody is a measure of the strength of the binding between antibody and antigen, such that a low-affinity antibody binds weakly and high-affinity antibody binds firmly. The affinity of an antibody for a target antigen can be assessed according to the methods provided herein by varying the concentration of the antibody of interest or the expression level of the target antigen (e.g., using CRISPRi-seq, a variant of Perturb-seq, to allow transcriptional knockdown of the target antigen coupled to expression of a unique guide RNA barcode).

### B. Epitope Mapping and LIBRA-Seq

In some embodiments, a population of cells (e.g., a yeast expression library) disclosed herein can be used for epitope mapping. In some embodiments, the epitopes can be linked to a membrane protein for cell surface expression. In some embodiments, a yeast expression library can be generated such that each of a plurality of cells (or each of a subset of cells from the plurality of cells) expresses a different epitope of the same antigen. In some embodiments, the epitopes may be non-overlapping. In some embodiments, the epitopes may be partially overlapping. In some embodiments, the same antigen sequence is tiled into a plurality of overlapping subsequences, e.g., short overlapping subsequences of length k (k-mers). See, e.g., Paull et al., “A general approach for predicting protein epitopes targeted by antibody repertoires using whole proteomes,” PLoS One. 2019; 14(9): e0217668.

In some embodiments, the one or more epitopes displayed by the yeast display library is a series of overlapping epitopes that correspond to greater than about 90% of the amino acid sequence of the protein. In some embodiments, the one or more epitopes displayed by the yeast display library is a series of overlapping epitopes that correspond to greater than about any one of: 50%, 60%, 70%, 80%, 90%, or 95% of the amino acid sequence of the protein. In some embodiments, the one or more epitopes displayed by the yeast display library is a series of overlapping epitopes that correspond to 100% of the amino acid sequence of the protein. In some embodiments, the combined amino acid sequences of all the epitopes overlaps with about any one of: 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the amino acid sequence of the protein. In some embodiments, each amino acid of about 80% of the amino acid sequence of the protein overlaps with at least two epitopes displayed by the yeast display library. In some embodiments, each amino acid of about 80% of the amino acid sequence of the protein overlaps with at least three epitopes displayed by the yeast display library. In some embodiments, each amino acid of about 90% of the amino acid sequence of the protein overlaps with at least two epitopes displayed by the yeast display library. In some embodiments, each amino acid of about 90% of the amino acid sequence of the protein overlaps with at least three epitopes displayed by the yeast display library. In some embodiments, each amino acid of about 95% of the amino acid sequence of the protein overlaps with at least two antigens derived from the protein. In some embodiments, each amino acid of about 95% of the amino acid sequence of the protein overlaps with at least three epitopes displayed by the yeast display library. In some embodiments, each of a plurality of yeast cells (or each of a subset of the plurality of yeast cells) could be engineered to an epitope variant from an epitope variant panel, for fine mapping of antigen specificity. For example, a first round of epitope mapping can be used to identify a broader region as epitopes, and a second round can use epitope variants/mutations at different residues to pinpoint residues, sequences, and/or structures involved in antigen specificity.

In some embodiments, the antibody (e.g., a monoclonal antibody) is engineered to comprise a nucleic acid barcode sequence, e.g., to assay its binding specificity for one or more binding partners and/or to distinguish it from other antibodies coupled to other nucleic acid barcode sequences. The antibody (e.g., a monoclonal antibody) may be engineered, e.g., fused to multiple different protein constructs to enable labeling. In some embodiments, the antibody is contacted with any one of the yeast cell libraries described herein. In some embodiments, the antibody binds to a yeast cell of a library via one or more molecules of an antigen or epitope exogenously expressed by the yeast cell, wherein the yeast cell comprises a nucleic acid sequence (e.g., barcode sequence) corresponding to the antigen or epitope, e.g., uniquely identifying the antigen or epitope from among the plurality of antigens or epitopes expressed by yeast cells of the library.

In some embodiments, antibody-bound yeast cells may be enriched, purified, isolated, sorted, and/or separated (e.g., from one or more cells not bound by an antibody or antigen-binding fragment thereof in the sample). In some embodiments, one or more yeast cells not bound by an antibody or antigen-binding fragment thereof in the sample are enriched, purified, isolated, sorted, and/or separated (e.g., from one or more antibody-bound yeast cells).

In some embodiments, antibody-bound yeast cells are partitioned into emulsion droplets and a partition may comprise a bead that comprises molecules comprising a barcode sequence (e.g., a partition-specific barcode sequence), which may be different from the barcode sequence identifying the antigen or epitope of interest of the yeast cell in the partition. In some embodiments, cells, e.g., yeast cells, may be lysed inside the partitions to release intracellular contents and the two types of barcodes, one identifying the antigen or epitope and other identify the partition (e.g., gel bead in the emulsion droplet) and the single yeast cell in the partition. Reverse transcription, DNA amplifications, and/or sequencing may be performed for identification of the displayed antigens and other cellular (e.g., intracellular, cell surface, secreted, soluble, or extracellular) analytes. In some aspects, the specificity of the reporter barcode labeled monoclonal antibody to the yeast epitope library may be identified and quantified, and correspondingly epitope mapping of the antibody can be analyzed.

In some embodiments, a method disclosed herein is used to screen a library of candidate antigens or epitopes, and the selected antigens or epitopes can be used in a LIBRA-seq (linking B cell receptor to antigen specificity through sequencing), a technology for high-throughput mapping of paired heavy- and light-chain BCR sequences to their cognate antigen specificities.

In some embodiments, a method disclosed herein is used to perform a LIBRA-seq (linking B cell receptor to antigen specificity through sequencing), a technology for high-throughput mapping of paired heavy- and light-chain BCR sequences to their cognate antigen specificities. In some embodiments, B cells are mixed with a library of nucleic acid-barcoded cells (such as yeast cells) with each cell displaying a uniquely-barcoded antigen so that upon isolation of cells that were bound by B cells, both the antigen-associated barcode(s) and BCR sequence can be recovered via single-cell next-generation sequencing. In some embodiments, wherein the cells further contain a fluorophore tag, both the antigen-binding B cells and the B cell-bound, antigen-displaying yeast cell carrying the barcode could be separated by flow cytometry. In some embodiments, the antigen-binding B cells can be partitioned into oil droplets with gel beads encoded by a separate oligonucleotide barcode and enzymes that drive a reverse transcription reaction. In some embodiments, both cellular BCR transcripts and the antigen-associated barcodes are captured by bead-delivered oligos, enabling direct mapping of BCR sequence to antigen specificity following sequencing. In some embodiments, B cells are isolated from peripheral blood. In some embodiments, B cells are isolated from peripheral blood mononuclear cells (PBMCs).

### III. Cells and Cell Populations

Cell surface display systems can express a protein or peptide on the surface of prokaryotic or eukaryotic cells (e.g., bacteria, yeast, insect, and mammalian cells). The protein or peptide can, for example, be coupled to a protein present at a cell surface and, by association with the cellular protein, can be displayed at the surface of the cell. Typically, the genetic information encoding the peptide or protein for display can be introduced into the cell (e.g., bacteria, yeast, insect, or mammalian cell) in the form of a polynucleotide element, such as a plasmid. Any suitable delivery method can be used for introducing a polynucleotide element, e.g., plasmid, into a cell. Non-limiting examples of delivery methods include, for example, viral or bacteriophage infection, transfection, conjugation, protoplast fusion, lipofection, electroporation, calcium phosphate precipitation polyethyleneimine (PEI)-mediated transfection, DEAE-dextran mediated transfection, liposome-mediated transfection, particle gun technology, calcium phosphate precipitation, direct microinjection, use of cell permeable peptides, and nano-particle mediated nucleic acid delivery. Conventional viral and non-viral based gene transfer methods can be used. Non-viral vector delivery systems can include DNA plasmids, RNA, naked nucleic acid, and nucleic acid complexed with a delivery vehicle, such as a liposome. Viral vector delivery systems can include DNA and RNA viruses, which can have either epiosomal or integrated genomes after delivery to the cell. Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, and agent-enhanced uptake of DNA. Cationic and neutral lipids that are suitable for efficient receptor-recognition lipofection of polynucleotides can be used. The preparation of lipid:nucleic acid complexes, including targeted liposomes such as immunolipid complexes, can be used. In some cases, expressing the peptide or protein comprises editing a cell genome via an integrase, recombinase, or Cas protein.

The cell can use the exogenous genetic information to produce the protein or peptide to be displayed. The genetic information (e.g., sequence-based information) can later be interrogated, for example by sequencing analysis, to determine the identity of a protein or peptide (e.g., amino acid sequence) identified in a binding assay or an interaction assay.

In an example, the coding sequence of a protein or peptide of interest can be linked to the coding sequence of a yeast cell wall protein. A non-limiting example of such a yeast protein is Aga2p which is used by yeast to mediate cell-cell contacts during yeast cell mating. The protein or peptide of interest can be tethered to the yeast cell wall protein, allowing the protein or peptide of interest to be displayed on the yeast cell surface. The protein or peptide displayed on the yeast cell surface can then be subjected to binding or interaction assays, and binding interactions of the protein or peptide can be studied by capturing the DNA or RNA sequence encoding the recombinantly displayed protein or peptide. In some cases, the DNA or RNA sequence can comprise a barcode sequence which specifically identifies the displayed protein or peptide. Similar systems are available for bacteria, insect cells, and mammalian cells. In cases where the protein or peptide binds to a cell or a component of a cell, information about the cell (e.g., transcriptome analysis, genome analysis, etc.) can also be obtained using methods disclosed herein.

In some cases, a library of cell-surface displayed proteins (e.g., yeast displayed) generated according to embodiments herein can be subjected to binding or interaction assays to identify proteins or peptides having certain properties of interest, for example, binding specificity, binding affinity, and biological activity. The library can include a plurality of proteins or peptides having different amino acid sequences displayed on a cell surface. Each member of the library can have unique biochemical or biophysical properties which can be analyzed by screening the library.

In some cases, the surface is not a cell surface. Non-limiting examples of technologies that do not utilize cells include phage display, mRNA display, and ribosome display. A protein of interest can be displayed, for example, on a phage by inserting the protein coding sequence into a phage coat protein gene. When the phage DNA is expressed as phage proteins, it can display the protein of interest on the surface of the phage, and package the corresponding DNA inside the phage capsid. The protein displayed on phage can then be subjected to binding or interaction assays, and binding interactions of the protein can then be studied by sequencing the phage DNA or mRNAs or by secondary labelling of the phage. In some cases, the phage DNA or mRNA includes a barcode sequence which is useful in identifying the protein of interest. In cases where the protein binds to a cell or a component of a cell, information about the cell (e.g., transcriptome analysis, genome analysis, etc.) can also be obtained using methods disclosed herein.

In some cases, a library of phage displayed proteins generated according to embodiments herein can be subjected to binding or interaction assays to identify proteins or peptides having certain properties of interest, for example, binding specificity, binding affinity, and biological activity. The library can include a plurality of proteins or peptides having different amino acid sequences displayed on phage. Each member of the library can have unique biochemical or biophysical properties which can be analyzed by screening the library.

In some embodiments, a protein of interested is produced by mRNA display for binding or interaction assays. In mRNA display, a translated protein can be associated with its coding mRNA via a linkage, e.g., a puromycin linkage. The protein of interest, linked to its coding mRNA, can then be subjected to binding or interaction assays, and binding interactions of the protein of interest can be studied by sequencing the coding mRNA, or a derivative thereof (e.g., cDNA transcript) linked to the protein. In some cases, the coding mRNA may be linked to a barcode sequence which can be used to identify the protein of interest. In cases where the protein binds to a cell or a component of a cell, information about the cell (e.g., transcriptome analysis, genome analysis, etc.) can also be obtained using methods disclosed herein.

In some cases, a library of mRNA displayed proteins generated according to embodiments herein can be subjected to binding or interaction assays to identify proteins or peptides having certain properties of interest, for example, binding specificity, binding affinity, and biological activity. The library can include a plurality of proteins or peptides having different amino acid sequences, each linked to its corresponding mRNA. Each member of the library can have unique biochemical or biophysical properties which can be analyzed by screening the library.

In some embodiments, a protein of interest is produced by ribosome display for binding or interaction assays. In ribosome display, the translated protein can be associated with its coding mRNA and a ribosome. The protein of interest, linked to its coding mRNA and a ribosome, can then be subjected to binding or interaction assays, and binding interactions of the protein can then be studied by sequencing the coding mRNA, or a derivative thereof (e.g., cDNA transcript) associated with the protein. In some cases, the coding mRNA may be linked to a barcode sequence which can be used to identify the protein of interest. In cases where the protein binds to a cell or a component of a cell, information about the cell (e.g., transcriptome analysis, genome analysis, etc.) can also be obtained using methods disclosed herein.

In some cases, a library of ribosome displayed proteins generated according to embodiments herein can be subjected to binding or interaction assays to identify proteins or peptides having certain properties of interest, for example, binding specificity, binding activity, and biological activity. The library can include a plurality of ribosome-displayed proteins or peptides having different amino acid sequences. Each member of the library can have unique biochemical or biophysical properties which can be analyzed by screening the library.

In an example, a method for using displayed proteins in a binding or interaction assay may comprise one or more of the following operations. A sample comprising immune cells (e.g., blood or a fraction thereof), preferably B cells, are mixed with a population displayed proteins (e.g., yeast-surface displayed, mammalian cell surface displayed, phage displayed, ribosome displayed, mRNA displayed, etc.) and incubated to allow for the immune cells and displayed proteins to interact. In some cases, the immune cell is a B cell and the B cell receptor (BCR) binds to a displayed protein. A B cell receptor can bind to a folded or unfolded polypeptide. The immune cells and displayed proteins can be partitioned such that bound BCR and displayed proteins are co-partitioned into the same partition (e.g., droplet, well, microwell, tube, etc.). Each of the partitions can also include a gel bead comprising one or more types of oligonucleotides. The oligonucleotide(s) attached to the bead can comprise a plurality of sequence elements useful for generating amplification products according to embodiments herein. For example, the oligonucleotide can comprise a barcode sequence (e.g., a partition specific barcode sequence), a unique molecular identifier sequence (UMI), and hybridization sequences (e.g., for primer extension). Within a partition, the immune cell can be lysed. If the protein display method employed includes a cell, e.g., a yeast cell or a mammalian cell, the display cell may also be lysed within the partition. For individual pairs of interacting B cells and displayed proteins, the identity of the protein (e.g., amino acid sequence) and identity of the B cell receptor (BCR) (e.g., receptor sequence) can be determined by sequencing nucleic acids derived therefrom. The coding mRNA of proteins of interest can be obtained and translated into a corresponding amino acid sequence. In cases where a barcode sequence is used, the polynucleotide sequence of the barcode itself can serve as an identifier. The sequence of the BCR can be obtained. Partition specific barcode sequences can be used to label and identify amplification products originating from common partitions (e.g., co-partitioned B cells and displayed proteins).

### A. Genetic Engineering of Cells

In some embodiments, the cell population to be engineered is a cell type commonly used for protein expression, such as yeast cell, a mammalian cell, e.g., Chinese hamster ovary (CHO) cells, baby hamster kidney (BHK) cells, NS0 myeloma and Sp2/0 hybridoma mouse cell lines, human embryonic kidney cells 293 (HEK293) and HT-1080 human cells. Human cell lines used or developed for biopharmaceutical protein production include HEK293, PER.C6, CEVEC’s amniocyte production (CAP), AGE1.HN, HKB-11 and HT-1080 cells. Several derivatives of HEK293 cells, such as HEK293-T and HEK293-EBNA1, have been developed from the parental HEK293 cells for improved recombinant protein production. Other nonhuman mammalian expression systems include the BHK-21 cells, murine NS0 myeloma and Sp2/0 hybridoma cells.

In some embodiments, a cell population is engineered to express a target antigen. In some embodiments, the cell population is engineered to express the target antigen via transient transfection or transduction, or via stable integration of an expression construct. In some embodiments, the expression construct can be delivered via any suitable vector (e.g., a plasmid vector or a viral vector such as an AAV vector or lentiviral vector).

In some embodiments, particularly for “difficult to transfect” cells such as T cells, an RNAi or guide RNA can be delivered via attachment to a molecule known to be internalized by the cell population of interest. There are multiple mechanisms by which small oligonucleotides (e.g. interfering RNAs) or their cationic complexes can internalize into mammalian cells. These include phagocytosis, pinocytosis, clathrin- and caveolin-dependent endocytosis. In particular, a type of endocytosis called “macropinocytosis” mediates non-selective uptake of tiny molecules, such as viruses, bacteria, nanoparticles, nutrients and antigens. Macropinocytosis is initiated from cell surface membrane ruffles that fold back onto themselves forming heterogeneous-sized endocytic structures known as macropinosomes. In some embodiments, the short oligonucleotide can be internalized into a cell population (e.g., T-cells) through a macropinocytosis-like endocytic mechanism e.g. in the absence of transfection reagents or electroporation.

In some embodiments, an expression construct for a target antigen can be knocked-in to a specific locus using any suitable method. In some embodiments, a knock-in cell line can be generated using a CRISPR/Cas system using a guide RNA and a homology donor cassette comprising the expression construct.

In some embodiments, an engineered cell line can be a cell line engineered to stably express a Cas nuclease (e.g., Cas9).

In some embodiments, an engineered cell population comprises a knock-down or knockout (KO) cell line. In some embodiments, the gene knocked down or knocked out encodes a target antigen or epitope, or a candidate target antigen or epitope. In some embodiments, gene knock down can be achieved using siRNA or a CRISPRi system. In some embodiments, KO cell lines are generated via CRISPR-Cas9 and certified via Sanger sequencing. This type of KO cell line provides a complete loss-of-function phenotype from a single allele KO and eliminates any masking of the knockout from a second allele seen in diploid cell models. In some embodiments, the CRISPR-Cas9 system can be used for knocking out gene expression in vivo or in vitro by using a combination of an sgRNA (single guide RNA) along with Cas9 (dCas9) nuclease. In some embodiments, the CRISPR guide RNA and/or Cas9 expression constructs can be delivered via a lentiviral-based CRISPR system. Expression of the sgRNA and Cas9 are stable and can be used in dividing or non-dividing cells or whole model organisms. Lentiviruses are powerful tools for manipulating host cells because they allow stable genomic integration of engineered DNA in both dividing and non-dividing cells, multiply or in single copy.

In certain aspects, the cells can be engineered by delivering a compound or composition into a cell. In some embodiments, the compound is a single compound. In some embodiments, the compound is a mixture of compounds. In some embodiments, the compound comprises a nucleic acid. In some embodiments, the compound is a nucleic acid. Exemplary nucleic acids include, without limitation, recombinant nucleic acids, DNA, recombinant DNA, cDNA, genomic DNA, RNA, siRNA, mRNA, saRNA, miRNA, IncRNA, tRNA, and shRNA. In some embodiments, the nucleic acid is homologous to a nucleic acid in the cell. In some embodiments, the nucleic acid is heterologous to a nucleic acid in the cell. In some embodiments, the compound is a plasmid. In some embodiments, the compound comprises a protein or polypeptide. In some embodiments, the nucleic acid is a transposon. A transposon, or transposable element, is a DNA segment that inserts itself into another position within the genome.

In some embodiments, the compound is a protein or polypeptide. In some embodiments, the protein or polypeptide is a therapeutic protein, antibody, fusion protein, antigen, synthetic protein, reporter marker, or selectable marker. In some embodiments, the protein is a gene-editing protein or nuclease such as a zinc-finger nuclease (ZFN), transcription activator-like effector nuclease (TALEN), mega nuclease, or CRE recombinase.

### 1. Perturb-Seq

In some embodiments, provided herein is a method of analyzing the binding epitope of an antibody or antigen-binding fragment of interest, wherein the method comprises analyzing binding of the antibody or antigen-binding fragment of interest to one or more cells are engineered or otherwise modified to express candidate epitopes. In some embodiments, engineering of the cell populations comprises knocking down or knocking out a polynucleotide encoding, or controlling or regulating expression of, a candidate epitope. In some embodiments, the engineering comprises a CRISPR-mediated perturbation, such as CRISPR/Cas induced mutations, or CRISPR-based transcriptional interference (CRISPRi), which mediates gene inactivation with high efficacy and specificity (Qi et al, 2013; Gilbert et al., 2014; Horlbeck et al, 2016).

In some embodiments, engineering of the cell populations comprises introducing CRISPR-mediated perturbations combined with a cell barcoding strategy that encodes the identity of the CRISPR-mediated perturbation in an expressed transcript, such as using “Perturb-seq.” Perturb-seq has been described, for example in in WO2018119447; WO2019157529; WO2018112423A; and Replogle et al. Combinatorial single-cell CRISPR screens by direct guide RNA capture and targeted sequencing. Nat Biotechnol 38, 954-961 (2020), herein incorporated by reference in their entireties. The identity of the perturbation is encoded on an expressed guide barcode for each guide RNA. In some embodiments, the barcode sequences associated with each sgRNA are randomly assigned and unique. In some embodiments, the barcode sequences associated with each sgRNA are assigned by sequencing during library construction.

In some embodiments, a cell population can be infected with a pool of lentiviral constructs that encode sgRNAs and their associated barcodes. In some embodiments, the pool of lentiviral constructs comprises a library of barcoded guide vectors, e.g., vectors comprising a sgRNA targeting a candidate epitope. A library can comprise, at least 2 or more vectors. For example, a library can comprise at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3,000, 4,000, 5,000, 6,000, 7,000, 8,000, 9,000, 10,000 or more dual guide-vectors.

In the methods provided herein, a site-directed nuclease is expressed in the mammalian cells. In some examples, the mammalian cells stably express a site-directed nuclease. In some examples, the site-directed nuclease is constitutively expressed. In some examples, the site-directed nuclease is under the control of an inducible promoter. In some examples, the mammalian cells are infected with a vector comprising a polynucleotide sequence encoding the site-directed nuclease prior to or subsequent to infecting the cells with the plurality of vectors. In any of the methods described herein, the site-directed nuclease can be transiently or stably expressed in the mammalian cells. In some examples, the site- directed nuclease is encoded by an expression cassette in the cell, the expression cassette comprising a promoter operably linked to a polynucleotide encoding the site-directed nuclease. In some examples, the promoter operably linked to the polynucleotide encoding the site-directed nuclease is a constitutive promoter. In other examples, the promoter operably linked to the polynucleotide encoding the site-directed nuclease is inducible. For example, and not to be limiting, the site-directed nuclease can be under the control of a tetracycline inducible promoter, a tissue-specific promoter, or an IPTG-inducible promoter.

The methods described can be used with any site-directed nuclease that requires a constant region of an sgRNA for function. These include, but are not limited to RNA-guided site-directed nucleases. Examples include nucleases present in any bacterial species that encodes a Type II CRISPR/Cas system. For example, and not to be limiting, the site-directed nuclease can be a Cas9 polypeptide, a C2c2 polypeptide or a Cpfl polypeptide. In some examples, the site-directed nuclease is the site-directed nuclease is an enzymatically active site-directed nuclease, such as, for example, a Cas9 polypeptide. In some examples, the site- directed nuclease is a deactivated site-directed nuclease, for example, a dCas9 polypeptide.

In the methods provided herein, once the cells have been infected, the cells are cultured for a sufficient amount of time to allow sgRNA: site-directed nuclease complex formation and transcriptional modulation, such that a pool of cells expressing a detectable phenotype (e.g., binding or absence of binding to the antibody or antigen-binding fragment of interest) can be selected from the plurality of infected cells.

In some embodiments, the method comprises expressing a site-directed nuclease in the mammalian cells; exposing the cells to the antibody or antigen-binding fragment of interest, and separating a selected pool of cells that bind to the antibody or antigen-binding fragment of interest from the plurality of cells infected by the barcoded sgRNA library; and analyzing the sequences of the sgRNA barcode and the antibody or antigen-binding fragment barcode.

### B. Fixed Samples

In some embodiments, the engineered cell populations described herein may be subjected to conditions that allow antibody molecules or antigen binding fragments thereof from a sample to couple to intracellular molecules or analytes, such as intracellular antigens or epitopes. Such conditions may comprise fixation and permeabilization. Fixation of cells or constituents of cells may comprise application of a chemical species or chemical stimulus. The term “fixed” as used herein with regard to biological samples generally refers to the state of being preserved from decay and/or degradation. “Fixation” generally refers to a process that results in a fixed sample, and in some instances can include contacting biomolecules within a cell population with a fixative (or fixation reagent) for some amount of time, whereby the fixative results in covalent bonding interactions such as crosslinks between biomolecules in the cells. A “fixed cell population” may generally refer to a cell population that has been contacted with a fixation reagent or fixative. For example, a formaldehyde-fixed cell population has been contacted with the fixation reagent formaldehyde. Generally, fixed cells from a fixed cell population refer to cells that have been in contact with a fixative under conditions sufficient to allow or result in the formation of intra- and inter-molecular covalent crosslinks between biomolecules within the cell(s). Generally, contact of a cell with a fixation reagent (e.g., paraformaldehyde or PFA) results in the formation of intra- and inter-molecular covalent crosslinks between biomolecules within the cells(s). In some cases, provision of the fixation reagent, such as formaldehyde, may result in covalent aminal crosslinks within RNA, DNA, and/or protein molecules. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

In some embodiments, the fixative or fixation reagent useful in the methods of the present disclosure is formaldehyde. The term “formaldehyde” when used in the context of a fixative also refers “paraformaldehyde” (or “PFA”) and “formalin”, both of which are terms with specific meanings related to the formaldehyde composition (e.g., formalin is a mixture of formaldehyde and methanol). Thus, a formaldehyde-fixed biological sample may also be referred to as formalin-fixed or PFA-fixed. Protocols and methods for the use of formaldehyde as a fixation reagent to prepare fixed biological samples are well known in the art, and can be used in the methods and compositions of the present disclosure. For example, suitable ranges of formaldehyde concentrations for use in preparing a fixed cell population is 0.1 to 10%, 1-8%, 1-4%, 1-2%, 3-5%, or 3.5-4.5%. In some embodiments of the present disclosure the cells are fixed using a final concentration of 1% formaldehyde, 4% formaldehyde, or 10% formaldehyde. Typically, the formaldehyde is diluted from a more concentrated stock solution - e.g., a 35%, 25%, 15%, 10%, 5% PFA stock solution.

It is contemplated that more than one fixation reagent can be used in combination in preparing a fixed cell population. For example, in some cases the cell populations are contacted with a fixation reagent containing both formaldehyde and glutaraldehyde, and thus the contacted biomolecules can include fixation crosslinks resulting both from formaldehyde induced fixation and glutaraldehyde induced fixation. Typically, a suitable concentration of glutaraldehyde for use as a fixation reagent is 0.1 to 1%.

The engineered cell populations may be permeabilized prior to, simultaneously with, or after treatment with a fixative. Cells may be permeabilized to provide access to a plurality of intracellular molecules included therein. Intracellular molecules or analytes from different parts of a cell and/or a specific sub-cellular region can be targeted with permeabilization reagents. For example, permeabilization reagents can be selected to increase the accessibility (e.g., by an antibody molecule from a sample) to analytes from the cytosol, from cell nuclei, from mitochondria, from microsomes, and more generally, from any other compartment, organelle, or portion of a cell. Permeabilizing agents that specifically target certain cell compartments and organelles can be used to selectively target analytes from cells for analysis. Permeabilization may involve partially or completely dissolving or disrupting a cell membrane or a portion thereof. Permeabilization may be achieved by, for example, contacting a cell membrane with an organic solvent or a detergent such as saponin, Triton X-100™, Tween-20™, or sodium dodecyl sulfate (SDS). Other suitable agents for this purpose include, but are not limited to, organic solvents (e.g., acetone, ethanol, and methanol), cross-linking agents (e.g., paraformaldehyde), and enzymes (e.g., trypsin, proteases (e.g., proteinase K). See, e.g., U.S. Pub. No. 20200277663, herein incorporated by reference in its entirety).

Upon binding by antibody molecules or antigen fragments thereof from a sample to intracellular molecules in engineered cells that have been fixed and optionally permeabilized, the engineered cells may be further processed as described herein, e.g., enrichment, coupling of a reporter molecule, partitioned for single cell analysis, etc.

### IV. Labels and Barcodes

An antibody, an antigen or epitope, a cell (e.g., a yeast cell), and/or other analytes disclosed herein may be labeled by a labelling agent. The labelling agents described herein may include, but are not limited to, an antibody or antibody fragment, a cell surface receptor binding molecule, a cell surface protein, a receptor ligand, a small molecule, a bi-specific antibody, a bi-specific T-cell engager, a T-cell receptor engager, a B-cell receptor engager, a pro-body, ribozyme, a monobody, an affimer, a darpin, and a protein scaffold. The labelling agents may have binding affinity for one or more analytes (e.g., proteins). The labelling agents may have binding affinity for one or more proteins based on the presence or absence of one or more posttranslational modifications, such as phosphorylation, glycosylation, ubiquitination, methylation, or acetylation. For example, a labelling agent (e.g., an antibody or antibody fragment) may have binding affinity for a protein when phosphorylated at one or more specific sites (e.g., may be a phosphospecific antibody). The labelling agents may be coupled, through the coupling approaches as described herein, to a reporter oligonucleotide comprising a nucleic acid barcode sequence that permits identification of the labelling agent, as described herein. In some embodiments, the nucleic acid barcode sequence coupled to the labelling agent comprises a unique molecular identifier (UMI) sequence segment, as described herein. The labelling agents described herein may also include fatty acids, cholesterol, or other cell membrane intercalating agents that can be used to associate DNA barcodes with an analyte. In some embodiments, the labelling agent is a lipid-displaying molecule (e.g., a CD1d protein or polypeptide) that can be utilized to label analytes such as cell receptors specific for the displayed lipid.

In some embodiments, the labelling agent is a small molecule binding agent (e.g., biotin, folic acid, or any suitable chemical entities capable of binding or interacting with a protein, DNA, or other biomolecule). Small molecule binding agents can be barcoded by chemical linkage to oligonucleotide barcodes for use as primary labelling agents or can be unlabeled with the analyte detected by a secondary barcoded labelling agent that binds or interacts with the primary unlabeled small molecule.

In some embodiments, the labelling agent is an aptamer. Aptamers are single stranded oligonucleotides that fold into a 3-D shape and are capable of binding small molecules such as toxins, antibiotics, heavy metals, and proteins. In some embodiments, aptamers utilized as labelling agents are directly or indirectly coupled with a barcode, e.g., directly in the aptamer sequence or indirectly through hybridization, ligation, or functionalization of the aptamer (e.g., with biotin).

The labelling agents described herein may not interact directly with the analyte, but rather function as a secondary labelling agent. For example, a first agent (e.g., a primary antibody) that does not comprise a barcode oligonucleotide may bind or couple to an analyte (e.g., a cell surface feature) and a secondary labelling agent (e.g., a secondary antibody or antibody binding protein) comprising a barcode oligonucleotide becomes associated or coupled to the analyte through interaction with the first agent, e.g., primary antibody. Exemplary affinities for the secondary antibody include, but are not limited to fluorophores (e.g., anti-phycoerythrin) and species-binding antibodies (e.g., goat, anti-mouse secondary antibody). In some embodiments, the labelling agent comprising the barcode oligonucleotide interacts with the analyte through a tertiary, quaternary, or larger interaction.

Multiple types of the labelling agents described herein may be used simultaneously to characterize an analyte (e.g., a barcoded antibody and a barcoded antigen or epitope, mRNA display together with fatty acid labelling).

In some embodiments, the analytes (e.g., an antibody-coated cell, or a cell comprising a labelling agent bound to a cell surface receptor) can be physically sorted. Physical cell sorting can be paired with a variety of approaches, such as associating a fluorophore or other detectable molecule (radioactive molecule, etc.) with a labelling agent and/or display techniques discussed herein. Cells can then be physically sorted by flow cytometry such that only cells with desired phenotypes are partitioned for analyte characterization. For example, a non-barcoded fluorescent molecule (e.g., PE-streptavidin) can be used to create a fluorescent and barcoded antigen or epitope as described herein. A sample comprising antibodies (e.g., serum or plasma) would be incubated with the fluorescent and barcoded antigen or epitope present in or on a yeast cell and then sorted with flow cytometry to isolate the subset of yeast cells with antigens or epitopes which have affinity for one or more antibodies in the sample. These antibody-coated cells are then partitioned and sequenced as generally described herein resulting in the identification of antigen binding specificities by antibodies in the sample.

In some embodiments, a protein or peptide used in a binding or interaction assay to characterize or detect an analyte may not comprise a physical label but can instead be associated with sequence-based information useful in identifying the protein or peptide. In some cases, a protein or peptide can be displayed on a surface for a binding assay or an interaction assay. The protein or peptide, in some embodiments, can be displayed on a cell surface using cell surface display systems. In some cases, a protein or peptide displayed on a surface for a binding assay is the analyte to be characterized. In other cases, the analyte to be characterized is the interacting or binding partner of the protein or peptide displayed on a surface. In some instances, the protein or peptide displayed on a surface and the interacting or binding partner of the displayed protein or peptide are both the analytes to be characterized.

The methods described herein may compartmentalize (e.g., partition) the analysis of individual cells or small populations of cells, including e.g., cell surface features, proteins, and nucleic acids of individual cells or small groups of cells, and then allow that analysis to be attributed back to the individual cell or small group of cells from which the cell surface features, proteins, and nucleic acids were derived. This can be accomplished regardless of whether the cell population represents a 50/50 mix of cell types, a 90/10 mix of cell types, or virtually any ratio of cell types, as well as a complete heterogeneous mix of different cell types, or any mixture between these.

Unique identifiers, e.g., barcodes, may be previously, subsequently, or concurrently delivered to the partitions that hold the compartmentalized or partitioned cells, in order to allow for the later attribution of the characteristics of the individual cells to the particular compartment. Further, unique identifiers, e.g., barcodes, may be coupled to the analytes and previously, subsequently, or concurrently delivered to the partitions that hold the compartmentalized or partitioned cells, in order to allow for the later attribution of the characteristics of the individual cells to the particular compartment.

In some embodiments, a given partition comprises a plurality of oligonucleotides comprising a barcode sequence, wherein said plurality of oligonucleotides are identical, and wherein said plurality of oligonucleotides are capable of coupling to two or more analytes (e.g., an mRNA molecule and an adapter sequence of a labelling agent). In some embodiments, a given partition comprises (a) a first plurality of oligonucleotides comprising a first barcode sequence; and (b) a second plurality of oligonucleotides comprising a second barcode sequence; wherein said first plurality of oligonucleotides are capable of coupling to a first analyte (e.g., gDNA, processed gDNA (e.g., ATAC-seq, DNase-seq, MNase-seq, etc,) and wherein said second plurality of oligonucleotides are capable of coupling to a second analyte (e.g., mRNA). In some embodiments, said first plurality of oligonucleotides comprise a first capture sequence (e.g., a random N-mer or ATAC-seq oligonucleotide as disclosed herein) and said second plurality of oligonucleotides comprise a second capture sequence (e.g., a poly-T sequence).

In other embodiments, a given partition comprises (a) a first plurality of oligonucleotides comprising a first barcode sequence; and (b) a second plurality of oligonucleotides comprising a second barcode sequence; wherein said first plurality of oligonucleotides are capable of coupling to a first analyte (e.g., a first adapter sequence present in, e.g., a CRISPR sgRNA molecule) and wherein said second plurality of oligonucleotides are capable of coupling to at least two additional analytes (e.g., an mRNA molecule and an adapter sequence of a labelling agent oligonucleotide, e.g., a barcode antigen or epitope, and/or a barcoded antibody). In some embodiments, said first plurality of oligonucleotides comprise a first capture sequence (e.g., a sequence complementary to an adapter sequence present in, e.g., a CRISPR sgRNA molecule) and said second plurality of oligonucleotides comprise a second capture sequence (e.g., a rGrGrG sequence complementary to a CCC sequence of a labelling agent oligonucleotide and a CCC sequence present on the 5′ end of a cDNA molecule).

In some embodiments, a given partition comprises (a) a first plurality of oligonucleotides comprising a first barcode sequence; (b) a second plurality of oligonucleotides comprising a second barcode sequence; and (c) a third plurality of oligonucleotides comprising a third barcode sequence; wherein said first plurality of oligonucleotides are capable of coupling to a first analyte (e.g., gDNA, processed gDNA (e.g., ATAC-seq, DNase-seq, MNase-seq, etc,), wherein said second plurality of oligonucleotides are capable of coupling to a second analyte (e.g., mRNA), and wherein said third plurality of oligonucleotides are capable of coupling to a third analyte (e.g., an adapter sequence of a labelling agent oligonucleotide, e.g., a barcode antigen or epitope, and/or a barcoded antibody). In some embodiments, said first plurality of oligonucleotides comprise a first capture sequence (e.g., a random N-mer or ATAC-seq oligonucleotide as disclosed herein), said second plurality of oligonucleotides comprise a second capture sequence (e.g., a poly-T sequence), and said third plurality of oligonucleotides comprise a third capture sequence (e.g., a sequence complementary to an adapter sequence of a labelling agent oligonucleotide, e.g., a barcode antigen or epitope, and/or a barcoded antibody).

In some embodiments, a given partition comprises (a) a first plurality of oligonucleotides comprising a first barcode sequence; (b) a second plurality of oligonucleotides comprising a second barcode sequence; and (c) a third plurality of oligonucleotides comprising a third barcode sequence; wherein said first plurality of oligonucleotides are capable of coupling to a first analyte (e.g., gDNA, processed gDNA (e.g., ATAC-seq, DNase-seq, MNase-seq, etc,), wherein said second plurality of oligonucleotides are capable of coupling to a second analyte (e.g., mRNA), and wherein said third plurality of oligonucleotides are capable of coupling to at least two additional analytes (e.g., an mRNA molecule and an adapter sequence of a labelling agent oligonucleotide, e.g., a barcode antigen or epitope, and/or a barcoded antibody). In some embodiments, said first plurality of oligonucleotides comprise a first capture sequence (e.g., a random N-mer or ATAC-seq oligonucleotide as disclosed herein), said second plurality of oligonucleotides comprise a second capture sequence (e.g., a poly-T sequence), and said third plurality of oligonucleotides comprise a third capture sequence (e.g., a rGrGrG sequence complementary to a CCC sequence of a labelling agent oligonucleotide and a CCC sequence present on the 5′ end of a cDNA molecule).

In some embodiments, a given partition comprises (a) a first plurality of oligonucleotides comprising a first barcode sequence and a first capture sequence; (b) a second plurality of oligonucleotides comprising a second barcode sequence and a second capture sequence; (c) a third plurality of oligonucleotides comprising a third barcode sequence and a third capture sequence; and (d) a fourth plurality of oligonucleotides comprising a fourth barcode sequence and a fourth capture sequence wherein said first plurality of oligonucleotides are capable of coupling to a first analyte (e.g., gDNA, processed gDNA (e.g., ATAC-seq, DNase-seq, MNase-seq, etc,), wherein said second plurality of oligonucleotides are capable of coupling to a second analyte (e.g., mRNA), wherein said third plurality of oligonucleotides are capable of coupling to a third analyte (e.g., an adapter sequence of a labelling agent oligonucleotide, e.g., a barcode antigen or epitope, and/or a barcoded antibody), and wherein said fourth plurality of oligonucleotides are capable of coupling to a fourth analyte (e.g., a first adapter sequence present in, e.g., a CRISPR sgRNA molecule). In other embodiments, a given partition comprises (a) a first plurality of oligonucleotides comprising a first barcode sequence and a first capture sequence; (b) a second plurality of oligonucleotides comprising a second barcode sequence and a second capture sequence; (c) a third plurality of oligonucleotides comprising a third barcode sequence and a third capture sequence; and (d) a fourth plurality of oligonucleotides comprising a fourth barcode sequence and a fourth capture sequence; wherein said first plurality of oligonucleotides are capable of coupling to a first analyte, wherein said second plurality of oligonucleotides are capable of coupling to a second analyte, wherein said third plurality of oligonucleotides are capable of coupling to a third analyte, and wherein said fourth plurality of oligonucleotides are capable of coupling to at least two or more analytes.

As described herein, the bead may comprise a gel bead. Further, as described herein, the bead may comprise a diverse library of capture oligonucleotides (e.g., barcoded oligonucleotides capable of coupling to an analyte). In some instances, the bead may comprise at least about 1,000 copies of a capture oligonucleotide, at least about 10,000 copies of a capture oligonucleotide, at least about 100,000 copies of a capture oligonucleotide, at least about 100,000 copies of a capture oligonucleotide, at least about 1,000,000 copies of a capture oligonucleotide, at least about 5,000,000 copies of a capture oligonucleotide, or at least about 10,000,000 copies of a capture oligonucleotide. In some instances, the bead may comprise at least about 1,000 copies of diverse capture oligonucleotides, at least about 10,000 copies of diverse capture oligonucleotides, at least about 100,000 copies of diverse capture oligonucleotides, at least about 100,000 copies of diverse capture oligonucleotides, at least about 1,00,000 copies of diverse capture oligonucleotides, at least about 5,000,000 copies of diverse capture oligonucleotides, or at least about 10,000,000 copies of diverse capture oligonucleotides. In some instances, and as described herein, releasing capture oligonucleotides from the bead may comprise subjecting the bead to a stimulus that degrades the bead. In some instances, as described herein, releasing capture oligonucleotides from the bead may comprise subjecting the bead to a chemical stimulus that degrades the bead.

A solid support (e.g., a bead) may comprise different types of capture oligonucleotides for analyzing both intrinsic and extrinsic information of a cell. For example, a solid support may comprise one or more of the following: 1) a capture oligonucleotide comprising a primer that binds to one or more endogenous nucleic acids in the cell; 2) a capture oligonucleotide comprising a primer that binds to one or more exogenous nucleic acids in the cell, e.g., nucleic acids from a microorganism (e.g., a virus, a bacterium) that infects the cell, nucleic acids introduced into the cell (e.g., such as plasmids or nucleic acid derived therefrom), nucleic acids for gene editing (e.g., CRISPR-related RNA such as crRNA, guide RNA); 3) a capture oligonucleotide comprising a primer that binds to a barcode (e.g., a barcode of a nucleic acid, of a protein, or of a cell); and 4) a capture oligonucleotide comprising a sequence (e.g., a primer) that binds to a protein, e.g., an exogenous protein expressed in the cell, an protein from a microorganism (e.g., a virus, a bacterium) that infects the cell, or an binding partner for a protein of the cell (e.g., an antigen for an immune cell receptor).

Disclosed herein, in some embodiments, are compositions, methods, and systems useful in the analysis of multiple analytes in a single cell or cell population. Examples of analytes include, without limitation, DNA (e.g., genomic DNA), epigenetic information (e.g., accessible chromatin or DNA methylation), RNA (e.g., mRNA or CRISPR guide RNAs), synthetic oligonucleotides (e.g., DNA transgenes), and proteins (e.g., intracellular proteins, cell surface proteins or features, extracellular matrix proteins, or nuclear membrane proteins). Examples of intracellular protein analytes include, but are not limited to, transcription factors, histone proteins, kinases, phosphatases, cytoskeletal proteins (e.g., actin, tubulin), polymerases, nucleases, and ribosomal proteins. An analyte may be a cell or one or more constituents of a cell.

### A. Nucleic Acid Barcodes for Antigens or Epitopes and Antibodies

In some embodiments, a cell such as yeast cell is engineered or otherwise modified to express an antigen or epitope, and the cell comprises a nucleic acid molecule comprising a nucleic acid sequence corresponding to the antigen or epitope. In some embodiments, the nucleic acid sequence identifies the antigen or epitope. In some embodiments, the nucleic acid sequence comprises a barcode sequence (e.g., antigen barcode) uniquely identifying the antigen or epitope from among the plurality of antigens or epitopes expressed by cells of a library. In some embodiments, the nucleic acid sequence comprises a barcode sequence (e.g., antigen barcode) shared by a set of antigens or epitopes, and the barcode sequence uniquely identifies the set of antigens or epitopes from one or more other sets in the plurality of antigens or epitopes expressed by cells of a library. Thus, two or more antigens or epitopes may be identified by the same antigen barcode, e.g., when the two or more antigens or epitopes are structurally or functionally similar or otherwise associated.

In some embodiments, the population of cells are contacted with a sample comprising a plurality of antibody molecules or antigen-binding fragments thereof, one or more of which is coupled to a nucleic acid sequence, e.g., an antibody barcode sequence.

In some embodiments, the nucleic acid sequence (e.g., comprising a barcode sequence) is directly coupled to the antigen or epitope. In some embodiments, the nucleic acid sequence (e.g., comprising a barcode sequence) is indirectly coupled to the antigen or epitope. In some embodiments, the nucleic acid sequence (e.g., comprising a barcode sequence) is directly coupled to the antibody. In some embodiments, the nucleic acid sequence (e.g., comprising a barcode sequence) is indirectly coupled to the antibody.

In another embodiment, where a cell comprises a cell surface protein, e.g., a cell that has been engineered or otherwise modified to express an antigen or epitope, and one or more antibody molecules bound to the cell surface protein, a nucleic acid reporter molecule may be coupled to the one or more bound antibody molecules while still bound to the cell. In one embodiment, the nucleic acid reporter molecule may be coupled to an antibody molecule via its carbohydrate groups (e.g., glycosylation sites), for example, on the constant region (e.g., Fc) of the antibody molecule. As further described herein, click chemistry approaches could be used to couple nucleic acid reporter molecules via carbohydrate groups or glycans of the antibody’s constant region.

Attachment (coupling) of the nucleic acid sequence to the antigen or epitope and/or antibody may be achieved through any of a variety of direct or indirect, covalent or non-covalent associations or attachments. For example, oligonucleotides may be covalently attached to a portion of the antigen or epitope and/or antibody using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides (e.g., barcodes) to antigens or epitopes and/or antibodies. Commercially available kits, such as those from Thunderlink and Abeam, and techniques common in the art may be used to couple reporter oligonucleotides to the antigen or epitope and/or antibody as appropriate. In another example, the antigen or epitope and/or antibody is indirectly (e.g., via hybridization) coupled to a reporter oligonucleotide comprising a barcode sequence that identifies the antigen or epitope and/or antibody. For instance, the antigen or epitope and/or antibody may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the antigen or epitope and/or antibody to the reporter oligonucleotide. In some embodiments, the reporter oligonucleotides are releasable from the antigen or epitope and/or antibody, such as upon application of a stimulus. For example, the reporter oligonucleotide may be attached to the antigen or epitope and/or antibody through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an RI, R2, or partial R1 or R2 sequence).

In some embodiments, a yeast cell is engineered by introducing into the cell an expression system capable of expressing an antigen or epitope in the cell. In some embodiments, the expression system is introduced into the genome of the cell. In some embodiments, the expression system comprises a coding sequence for the antigen or epitope and optionally a tag such as an affinity tag (e.g., His-tag) to be fused to the antigen or epitope when expressed. In some embodiments, the coding sequence comprises or forms part of the nucleic acid sequence corresponding to the antigen or epitope. In some embodiments, all or a part of the coding sequence (or a complement thereof) is used to identify the antigen or epitope. For example, a transcript of the expression system comprising a sequence of the coding sequence can be coupled to a barcode nucleic acid molecule in a partition for subsequent analysis on a single-cell basis.

In some embodiments, the expression system comprises an antigen barcode sequence or a complement thereof corresponding to the antigen or epitope, wherein the antigen barcode sequence or complement thereof is distinct from a coding sequence for the antigen or epitope. In some embodiments, the expression system comprises a sequence or a complement thereof configured to couple to a barcode nucleic acid molecule in a partition. In some embodiments, a transcript comprising an antigen barcode sequence or a complement thereof can be coupled to a barcode nucleic acid molecule in a partition for subsequent analysis on a single-cell basis. In some embodiments, the transcript comprises a coding sequence for the antigen or epitope, an antigen barcode sequence or a complement thereof corresponding to the antigen or epitope, and a sequence or a complement thereof configured to couple the transcript to a barcode nucleic acid molecule in a partition.

### B. Cell Surface Labelling

In some embodiments of the methods provided herein, a cell surface protein can be labeled at the cell surface using proximity-based labeling with an oligonucleotide barcode. In some embodiments, proximity-based labeling can be used to detect binding of a binding molecule (e.g., an antibody or antigen binding fragment) to an antigen or target epitope. In some examples, a cell population expressing target antigens or epitopes can be engineered to express a secreted antibody of interest, and self-binding of the secreted antibody to the target antigen or epitope can be detected via sortase-mediated attachment of a nucleic acid barcode sequence to the target antigen or epitope.

### 1. Proximity-Based Labeling of a Secreted Antibody

In some embodiments, provided herein is a method of analyzing an antibody or antigen-binding fragment thereof, comprising: partitioning a cell into a partition with a barcode bead, wherein the cell is engineered to secrete an antibody or antigen-binding fragment thereof and express a candidate epitope, and upon secretion of the antibody or antigen-binding fragment thereof from the cell and binding to the candidate epitope, the antibody or antigen-binding fragment thereof is directly or indirectly coupled to a first nucleic acid barcode sequence, wherein the barcode bead comprises a second nucleic acid barcode sequence, wherein when the antibody or antigen-binding fragment thereof specifically binds the candidate epitope, a barcoded nucleic acid molecule is generated in the partition, wherein the barcoded nucleic acid molecule comprises (i) a sequence corresponding to the first nucleic acid barcode sequence and (ii) a sequence corresponding to the second nucleic acid barcode sequence, and wherein the barcoded nucleic acid molecule is analyzed to analyze the binding between the antibody or antigen-binding fragment thereof and the candidate epitope. Methods of labeling a target protein upon binding with a binding partner protein have been described. See, e.g., Pasqual et al. “Monitoring T cell-dendritic cell interactions in vivo by intercellular enzymatic labelling.” Nature vol. 553,7689 (2018): 496-500, which describes the Labelling Immune Partnerships by SorTagging Intercellular Contacts (LIPSTIC) method.

In some embodiments, the antibody or antigen-binding fragment thereof is covalently coupled to the first nucleic acid barcode sequence, e.g., via a sortase-catalyzed ligation. The protein comprising the candidate epitope can be fused to a sortase, e.g., a sortase capable of transferring its substrate onto a sortase acceptor peptide fused to the antibody or antigen-binding fragment of interest. Suitable sortases, sortase substrates (which comprise a sortase recognition sequence), and sortase acceptor peptides have been described, for example, in U.S. Pat. No. 10,053,683, herein incorporated by reference in its entirety. Selection of suitable combinations of the three components has been described.

Suitable sortases can include a sortase A, a sortase B, a sortase C, or a sortase D. In some embodiments, the sortase is a mutant SrtA that exhibits improved catalytic activity as compared to the wild-type counterpart. In some examples, the sortase is fused to the N-terminus of a member of a ligand-receptor pair. In other examples, the sortase is fused to the C-terminus of the member of the ligand-receptor pair.

In some embodiments, the antibody or antigen-binding fragment of interest is fused to a sortase acceptor peptide via methods known in the art, e.g., recombinant technology. A sortase acceptor peptide can be any peptide that provides a nucleophilic acyl group for accepting a sortase substrate (a peptide comprising a sortase recognition sequence as described herein). Such an acceptor peptide may contain up to about 50 amino acids, such as up to 40, 30, 20, 15, 10, or 5 amino acids. In some embodiments, the acceptor peptide is an oligoglycine or oligoalanine, such as a 1-5 glycine fragment or a 1-5 alanine fragment. In some examples, the oligoglycine consists of 3 or 5 glycine residues. In other examples, the oligoalanine consists of 3 or 5 alanine residues. In some embodiments, the sortase acceptor peptide is fused to the amino-terminal end of the antibody or antigen-binding fragment of interest.

The sortase substrate used in the methods described herein, which is conjugated to a detectable label comprising a first nucleic acid barcode sequence, can comprise any sortase recognition sequence as known in the art or disclosed herein. Selection of a suitable sortase recognition sequence would depend on the type of sortase used in the same methods. Suitable sortase recognition sequences include LPXTG, in which X is any amino acid residue. In some embodiments, the sortase recognition sequence is LPETG, which may be co-used with a mutant SrtA described above.

A plurality of cells engineered such that the cells secrete an antibody or antigen-binding fragment thereof and each cell expresses a candidate epitope can be incubated in the presence of a suitable sortase substrate, which is associated with a detectable label under conditions allowing for occurrence of the transpeptidation reaction catalyzed by the sortase to conjugate the labeled sortase substrate to the sortase acceptor peptide. The detectable label includes a first nucleic acid barcode sequence, such that when the antibody or antigen-binding fragment thereof specifically binds the candidate epitope, the sortase conjugates the first nucleic acid barcode sequence to the sortase acceptor peptide fused to the antibody or antigen-binding fragment of interest.

If the secreted antibody or antigen-binding fragment binds to the candidate epitope expressed by the engineered cell, the spatial proximity would allow the sortase fused to the candidate epitope to transfer the labeled sortase substrate onto the sortase acceptor peptide on antibody or antigen-binding fragment, thereby labeling the antibody or antigen-binding fragment with the first nucleic acid barcode sequence.

In some embodiments, a reporter barcode labeled binding molecule (e.g., antigen or epitope, or a monoclonal antibody or antigen-binding fragment thereof) is covalently coupled to a binding partner (e.g., an antigen or epitope) via a spontaneous isopeptide reaction between peptide tags. Proteins that are capable of spontaneous isopeptide bond formation have been used to develop peptide tag/binding partner pairs which covalently bind to each other and which hence provide irreversible interactions (see e.g. WO2011/098772 herein incorporated by reference). In some embodiments, the binding molecule (e.g., barcode labeled antibody) is labeled with a peptide tag and the binding partner (e.g. antigen) is labeled with a binding partner for the peptide tag, wherein the peptide tag and binding partner of the peptide tag are capable of spontaneous isopeptide bond formation, such that binding of the binding molecule and binding partner results in formation of an isopeptide bond between the two. The isopeptide bond formed by the peptide tag and binding partner pairs can be stable under conditions where non-covalent interactions would rapidly dissociate, e.g. over long periods of time (e.g. weeks), at high temperature (to at least 95° C.), at high force, or with harsh chemical treatment (e.g. pH 2-11, organic solvent, detergents or denaturants). Thus, cells expressing a secreted binding molecule that binds to an antigen expressed on the cell surface, will be labeled by the binding molecule and its attached reporter molecule (e.g., reporter oligonucleotide).

In brief, a peptide tag/binding partner pair may be derived from any protein capable of spontaneously forming an isopeptide bond (an isopeptide protein), wherein the domains of the protein are expressed separately to produce a peptide tag that comprises one of the residues involved in the isopeptide bond (e.g. a lysine) and a peptide binding partner that comprises the other residue involved in the isopeptide bond (e.g. an asparagine or aspartate). In some instances, one of the peptide tag or binding partner comprises one or more other residues required to form the isopeptide bond (e.g. a glutamate).

In some embodiments, the domains comprising the residues involved in isopeptide bond formation can be expressed separately, i.e. as three separate peptides (domains, modules or units). In this respect, the peptide tag comprises one of the residues involved in the isopeptide bond (e.g. a lysine), the peptide binding partner that comprises the other residue involved in the isopeptide bond (e.g. an asparagine or aspartate) and a third peptide comprises the one or more other residues involved in isopeptide bond formation but not involved in the isopeptide bond itself (e.g. a glutamate). Mixing all three peptides results in the formation of an isopeptide bond between the two peptides comprising the residues that react to form the isopeptide bond, i.e. the peptide tag and binding partner. Thus, the third peptide mediates the conjugation of the peptide tag and binding partner but does not form of the part resultant structure, i.e. the third peptide is not covalently linked to the peptide tag or binding partner. As such, the third peptide may be viewed as a protein ligase or peptide ligase. This is particularly useful as it minimises the size of the peptide tag and binding partner that need to be fused to the protein of interest, thereby reducing the possibility of unwanted interactions caused by the addition of the peptide tag or binding partner, e.g. misfolding.

Various proteins which are capable of spontaneously forming one or more isopeptide bonds (a so-called “isopeptide protein”) have been identified (e.g., SpyTag/SpyCatcher or SnoopTag/SnoopCatcher and may be modified to produce a peptide tag/binding partner pair and optionally a peptide ligase (e.g., SpyLigase or SnoopLigase), as discussed above. Further proteins that are capable of spontaneously forming one or more isopeptide bonds may be identified by comparing their structures with those of proteins which are known to spontaneously form one or more isopeptide bonds. Particularly, other proteins which may spontaneously form an isopeptide bond may be identified by comparing their crystal structures with those from known isopeptide proteins e.g. the major pilin protein Spy0128, and in particular comparing the Lys-Asn/Asp-Glu/Asp residues often involved in the formation of an isopeptide protein. Additionally, other isopeptide proteins may be identified by screening for structural homologues of known isopeptide proteins using the Protein Data Bank using standard database searching tools. The SPASM server may be used to target the 3D structural template of Lys-Asn/Asp-Glu/Asp of the isopeptide bond or isopeptide proteins may also be identified by sequence homology alone. A peptide tag and binding partner, SpyTag and SpyCatcher that react spontaneously to form an isopeptide bond, and a three part SpyTag/KTag/SpyLigase have been developed previously (WO2011/098772 and U.S. Pub. No. 20200115422; herein incorporated by reference in their entirety). Orthogonal systems such as SnoopTag/SnoopCatcher or SnoopTagJr, DogTag, and SnoopLigase have also been described (see e.g. U.S. Patent No. 10526379 and U.S. Pub. No. 20200115422, herein incorporated by reference in their entirety).

The binding activities of the thus identified polypeptides can be confirmed by a conventional binding assay, e.g., ELISA assay.

In some embodiments, the labeling system described herein can be applied to identify intercellular binding contacts, such as between a population of antibody-expressing cells (e.g., B cells or T cells) and a population of cells expressing a candidate epitope. Cells conjugated to the detectable label can then be isolated via a routine method, e.g., by cell sorting. The labeled cells thus identified can be further analyzed to identify the cell-surface antibody and binding partner based on the nucleic acid barcode label.

In some embodiments, the cells can be separated into partitions with a barcode bead, wherein when the antibody or antigen-binding fragment thereof specifically binds the candidate epitope, a barcoded nucleic acid molecule is generated in the partition, wherein the barcoded nucleic acid molecule comprises (i) a sequence corresponding to the first nucleic acid barcode sequence and (ii) a sequence corresponding to the second nucleic acid barcode sequence of the barcode bead, and wherein the dually barcoded nucleic acid molecule is analyzed to analyze the binding between the antibody or antigen-binding fragment thereof and the candidate epitope.

### C. Click Chemistry

As used herein, the term “click chemistry,” generally refers to reactions that are modular, wide in scope, give high yields, generate only inoffensive byproducts, such as those that can be removed by nonchromatographic methods, and are stereospecific (but not necessarily enantioselective). See, e.g., U.S. Pat. Pub. 2019/0100632 (now U.S. Pat. 10,590,244), U.S. Pat. Pub. 2019/0233878, and Angew. Chem. Int. Ed., 2001, 40(11):2004-2021, which are entirely incorporated herein by reference for all purposes.

In some cases, click chemistry can describe pairs of functional groups that can selectively react with each other in mild, aqueous conditions. An example of click chemistry reaction can be the Huisgen 1,3-dipolar cycloaddition of an azide and an alkynes, e.g., Copper-catalyzed reaction of an azide with an alkyne to form a 5-membered heteroatom ring called 1,2,3-triazole. The reaction can also be known as a Cu(I)-Catalyzed Azide-Alkyne Cycloaddition (CuAAC), a Cu(I) click chemistry or a Cu⁺ click chemistry. Catalyst for the click chemistry can be Cu(I) salts, or Cu(I) salts made in situ by reducing Cu(II) reagent to Cu(I) reagent with a reducing reagent (Pharm Res. 2008, 25(10): 2216-2230). Known Cu(II) reagents for the click chemistry can include, but are not limited to, Cu(II)-(TBTA) complex and Cu(II) (THPTA) complex. TBTA, which is tris-[(1-benzyl-1H-1,2,3-triazol-4-yl)methyl]amine, also known as tris-(benzyltriazolylmethyl)amine, can be a stabilizing ligand for Cu(I) salts. THPTA, which is tris-(hydroxypropyltriazolylmethyl)amine, can be another example of stabilizing agent for Cu(I). Other conditions can also be accomplished to construct the 1,2,3-triazole ring from an azide and an alkyne using copper-free click chemistry, such as by the Strain-promoted Azide-Alkyne Click chemistry reaction (SPAAC, see, e.g., Chem. Commun., 2011, 47:6257-6259 and Nature, 2015, 519(7544):486-90), each of which is entirely incorporated herein by reference for all purposes.

In some cases, the present disclosure also contemplates the use of click chemistry reactions resulting in chemical linkages that are not a 1,2,3-triazole. See, e.g., U.S. Pat. Pub. 2019/0233878, which is incorporated by reference in its entirety. A range of such click chemistry reactions useful for preparing biocompatible gels are well-known in the art. See e.g., Madl and Heilshom, “Bioorthogonal Strategies for Engineering Extracellular Matrices,” Adv. Funct. Mater. 2018, 28: 1706046, which is hereby incorporated by reference herein.

An example of a click chemistry reaction useful in the compositions and methods of the present disclosure that is copper-free and does not result in a 1,2,3-triazole linkage is an Inverse-electron demand Diels-Alder (IED-DA) reaction. (See e.g., Madl and Heilshom 2018.) As described elsewhere herein, in the IED-DA click chemistry reaction, the pair of click chemistry functional groups comprises a tetrazine group and a trans-cyclooctene (TCO) group, or a tetrazine group and a norbonene group. This reaction is copper free and results in a linkage comprising a dihydropyridazine group rather than a 1,2,3-triazole.

Other specific biorthogonal click chemistry reactions that are useful in the compositions and methods of the present disclosure, but which result in a chemical linkage other than a 1,2,3-triazole include a Diels-Alder reaction between a pair of furan and maleimide functional groups, a Staudinger ligation, and nitrile oxide cycloaddition. These click chemistry reactions and others are well-known in the art and described in e.g., Madl and Heilshom 2018. Accordingly, in some embodiments the copper-free click chemistry useful in forming crosslinked polymers of the present disclosure can be selected from: (a) strain-promoted azide/dibenzocyclooctyne-amine (DBCO) click chemistry; (b) inverse electron demand Diels-Alder (IED-DA) tetrazine/trans-cyclooctene (TCO) click chemistry; (c) inverse electron demand Diels-Alder (IED-DA) tetrazine/norbonene click chemistry; (d) Diels-Alder maleimide/furan click-chemistry; (e) Staudinger ligation; and (f) nitrile-oxide/norbonene cycloaddition click chemistry.

### V. Cells Bound by Binding Molecules

Antibody-coated cells can then be detected and/or separated by mechanisms that allow the differentiation of the antibody-coated cells from non-coated cells. Exemplary methods include, but are not limited to, fluorescent-activated cell sorting (FACS), magnetic-activated cell sorting (MACS) or buoyancy-activated cell sorting (BACS).

In some embodiments, one or more markers specific to each population (e.g., antigen⁺ guide⁻ and antigen⁺ guide⁺) of yeast cells may be assayed to ensure equal or substantially equal representation of the cell populations for downstream analysis.

In some embodiments, the antibody-coated cells or cells of one or more populations can be detected and/or selected by using fluorescent-activated cell sorting (FACS). In some embodiments, antibody-coated cells can be detected by a fluorochrome-labeled antibody recognizing the cell-bound antibodies, and subsequently analyzed using FACS. In some embodiments, antibody-coated cells can be detected by an antibody (e.g., a fluorochrome-labeled antibody, a magnetic bead-labeled antibody, a microbubble-labeled antibody, or a combination thereof) recognizing a tag or target of the cell-bound antibodies, and subsequently analyzed.

In some embodiments, the antibody-coated cells or cells of one or more populations can be detected and/or selected by using magnetic-activated cell sorting (MACS). In some embodiments, antibody-coated cells can be detected by a magnetic bead-labeled antibody recognizing the cell-bound antibodies, and subsequently analyzed using MACS. In some embodiments, antibody-coated cells can be detected by an antibody (e.g., a fluorochrome-labeled antibody, a magnetic bead-labeled antibody, a microbubble-labeled antibody, or a combination thereof) recognizing a tag or target of the cell-bound antibodies, and subsequently analyzed.

In some embodiments, the antibody-coated cells or cells of one or more populations can be detected and/or selected by using buoyancy-activated cell sorting (BACS). In some embodiments, antibody-coated cells can be detected by a microbubble-labeled antibody recognizing the cell-bound antibodies. In some embodiments, antibody-coated cells can be bound by a microbubble-labeled antibody, resulting in lower density in a buoyancy separation. In some embodiments, antibody-coated cells can be detected by a microbubble-labeled antibody recognizing the Fc region of the cell-bound antibodies, and subsequently analyzed using BACS. In some embodiments, antibody-coated cells can be detected by an antibody (e.g., a fluorochrome-labeled antibody, a magnetic bead-labeled antibody, a microbubble-labeled antibody, or a combination thereof) recognizing a tag or target of the cell-bound antibodies, and subsequently analyzed.

In some embodiments wherein the detectable label for each cell population comprises a detectable nucleotide sequence (e.g., an oligonucleotide barcode), the different cell populations need not be separated prior to partitioning and sequencing analysis. Multiple cell populations can be combined and processed in parallel as one single sample via cellular barcoding, allowing detection of the cell population label in each single cell partition. Following data acquisition, individual cells can be unmixed in silico and reassigned back to their initial samples via their unique barcode.

### VI. Partitioning

In one aspect, the methods and system described herein provide for the compartmentalization, depositing or partitioning of individual cells (e.g., antibody-coated cells after contacting with an antibody such as a monoclonal antibody) from a sample material containing cells, into discrete compartments or partitions (referred to interchangeably herein as partitions), where each partition maintains separation of its own contents from the contents of other partitions. In one aspect, the methods described herein comprise the compartmentalization, depositing or partitioning of nucleic acid molecules (e.g., genomic DNA and/or nucleic acid barcodes directly or indirectly attached to the cell surface) of one or more individual cells from a sample material containing cells, into discrete partitions, where each partition maintains separation of its own contents from the contents of other partitions.

In another aspect, the methods and system described herein provide for the compartmentalization, depositing or partitioning of individual cells from a sample material containing cells after at least one labelling agent or reporter agent has been bound to a cell surface feature of a cell, into discrete partitions, where each partition maintains separation of its own contents from the contents of other partitions. Identifiers including unique identifiers and common or universal tags, e.g., barcodes, may be previously, subsequently or concurrently delivered to the partitions that hold the compartmentalized or partitioned cells, in order to allow for the later attribution of the characteristics of the individual cells to one or more particular compartments. Further, identifiers including unique identifiers and common or universal tags, e.g., barcodes, may be coupled to labelling agents and previously, subsequently or concurrently delivered to the partitions that hold the compartmentalized or partitioned cells, in order to allow for the later attribution of the characteristics of the individual cells to one or more particular compartments. Identifiers including unique identifiers and common or universal tags, e.g., barcodes, may be delivered, for example on an oligonucleotide, to a partition via any suitable mechanism.

In some embodiments, a partition herein includes a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### A. Systems and Methods for Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. In some examples, the partitioned particle is a labelled cell such as an antibody-coated cell (e.g., a cell expressing an antigen that is bound by an antibody such as an mAb of interest), and cells of one or more populations for which the expression and/or presence of an antigen in the cells can be identified. In other examples, the partitioned particle may be a labelled cell engineered to secrete antibodies. The partition can be a droplet in an emulsion. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its macromolecular constituents, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

Microfluidic devices or platforms comprising microfluidic channel networks (e.g., on a chip) can be utilized to generate partitions such as droplets and/or emulsions as described herein. Methods and systems for generating partitions such as droplets, methods of encapsulating analyte carriers and/or analyte carriers in partitions, methods of increasing the throughput of droplet generation, and various geometries, architectures, and configurations of microfluidic devices and channels are described in U.S. Pat. Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials (e.g., secreted analytes). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Pat. Application Publication No. 2014/0155295, which is entirely incorporated herein by reference for all purposes. Emulsion systems for creating stable droplets in non-aqueous or oil continuous phases are described in, for example, U.S. Pat. Application Publication No. 2010/0105112, which is entirely incorporated herein by reference for all purposes.

In the case of droplets in an emulsion, allocating individual particles to discrete partitions may, in one non-limiting example, be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell, or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

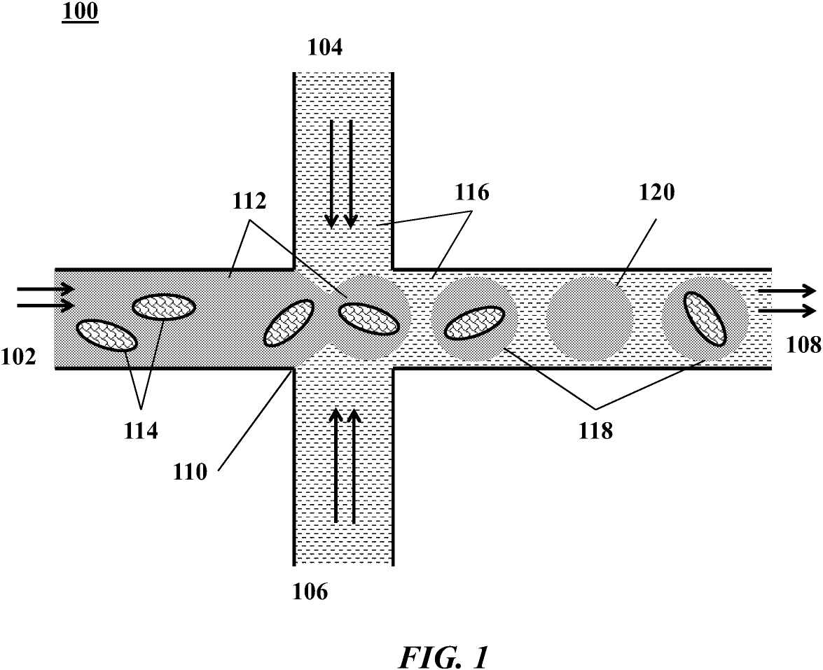

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (e.g., cells, i.e., labelled B cells or plasma cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Pat. Application Publication No. 2010/0105112, which is entirely incorporated herein by reference for all purposes.

As will be appreciated, the channel segments of the microfluidic devices described herein may be coupled to any of a variety of different fluid sources or receiving components, including reservoirs, tubing, manifolds, or fluidic components of other systems. As will be appreciated, the microfluidic channel structure 100 may have other geometries and/or configurations. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114 and (2) unoccupied droplets 120, not containing any biological particles 114. Occupied droplets 118 may comprise singly occupied droplets (having one biological particle) and multiply occupied droplets (having more than one biological particle, such as multiple B cells or plasma cells). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

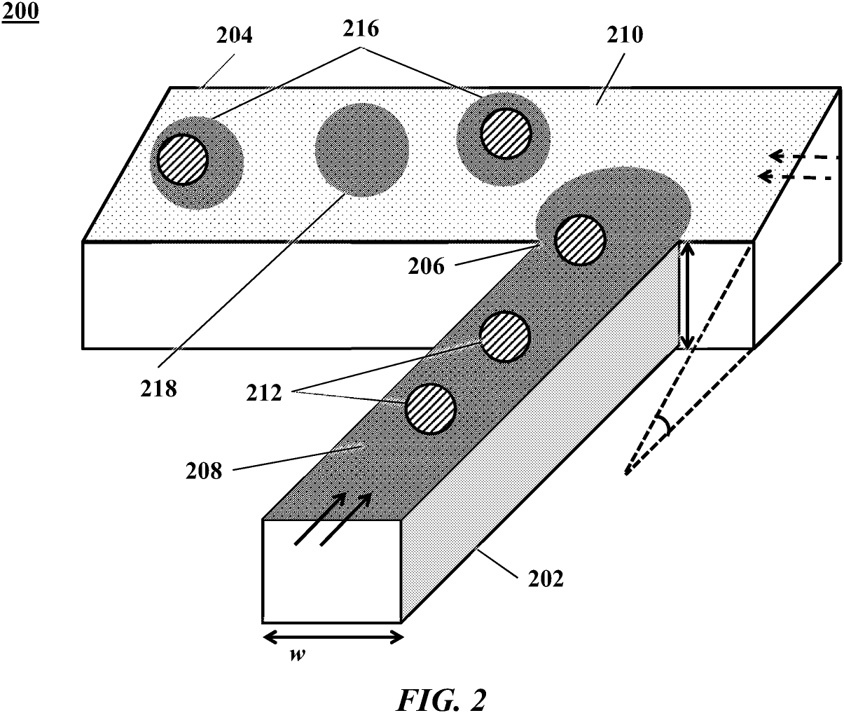

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides) (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Pat. Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles, or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Pat. Application Publication No. 2014/0378345, which is entirely incorporated herein by reference for all purposes.

The biological particle, can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents (e.g., secreted antibodies or antigen binding fragments thereof) of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized (e.g., coupled to a capture agent) to bind to targeted analytes (e.g., secreted antibodies or antigen binding fragment thereof), such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles or macromolecular constituents (e.g., RNA, DNA, proteins, secreted antibodies or antigen binding fragments thereof etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (e.g., cytokines, antigens, etc.). In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

### B. Samples and Cell Processing

A sample may be derived from any useful source including any subject, such as a human subject. A sample may comprise material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, may be obtained for analysis as described herein. For example, a first sample may be obtained from a subject at a first time and a second sample may be obtained from the subject at a second time later than the first time. The first time may be before a subject undergoes a treatment regimen or procedure (e.g., to address a disease or condition), and the second time may be during or after the subject undergoes the treatment regimen or procedure. In another example, a first sample may be obtained from a first bodily location or system of a subject (e.g., using a first collection technique) and a second sample may be obtained from a second bodily location or system of the subject (e.g., using a second collection technique), which second bodily location or system may be different than the first bodily location or system. In another example, multiple samples may be obtained from a subject at a same time from the same or different bodily locations. Different samples, such as different samples collected from different bodily locations of a same subject, at different times, from multiple different subjects, and/or using different collection techniques, may undergo the same or different processing (e.g., as described herein). For example, a first sample may undergo a first processing protocol and a second sample may undergo a second processing protocol.

A sample may be a biological sample, such as a cell sample (e.g., as described herein). A sample may include one or more analyte carriers, such as one or more cells and/or cellular constituents, such as one or more cell nuclei. For example, a sample may comprise a plurality of cells and/or cellular constituents. Components (e.g., cells or cellular constituents, such as cell nuclei) of a sample may be of a single type or a plurality of different types. For example, cells of a sample may include one or more different types of blood cells.

A biological sample may include a plurality of cells having different dimensions and features. In some cases, processing of the biological sample, such as cell separation and sorting (e.g., as described herein), may affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process may comprise the use of microfluidics (e.g., to separate analyte carriers of different sizes, types, charges, or other features).

In an example, a sample comprising one or more cells may be processed to separate the one or more cells from other materials in the sample (e.g., using centrifugation and/or another process). In some cases, cells and/or cellular constituents of a sample may be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process may comprise a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells may comprise, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method may be used to detect cells and/or cellular constituents based on a parameter such as a size, morphology, or protein expression. Flow cytometry-based cell sorting may comprise injecting a sample into a sheath fluid that conveys the cells and/or cellular constituents of the sample into a measurement region one at a time. In the measurement region, a light source such as a laser may interrogate the cells and/or cellular constituents and scattered light and/or fluorescence may be detected and converted into digital signals. A nozzle system (e.g., a vibrating nozzle system) may be used to generate droplets (e.g., aqueous droplets) comprising individual cells and/or cellular constituents. Droplets including cells and/or cellular constituents of interest (e.g., as determined via optical detection) may be labeled with an electric charge (e.g., using an electrical charging ring), which charge may be used to separate such droplets from droplets including other cells and/or cellular constituents. For example, FACS may comprise labeling cells and/or cellular constituents with fluorescent markers (e.g., using internal and/or external biomarkers). Cells and/or cellular constituents may then be measured and identified one by one and sorted based on the emitted fluorescence of the marker or absence thereof. MACS may use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS may use microbubbles (e.g., glass microbubbles) labeled with antibodies to target cells of interest. Cells and/or cellular components coupled to microbubbles may float to a surface of a solution, thereby separating target cells and/or cellular components from other components of a sample. Cell separation techniques may be used to enrich for populations of cells of interest (e.g., prior to partitioning, as described herein). For example, a sample comprising a plurality of cells including a plurality of cells of a given type may be subjected to a positive separation process. The plurality of cells of the given type may be labeled with a fluorescent marker (e.g., based on an expressed cell surface marker or another marker) and subjected to a FACS process to separate these cells from other cells of the plurality of cells. The selected cells may then be subjected to subsequent partition-based analysis (e.g., as described herein) or other downstream analysis. The fluorescent marker may be removed prior to such analysis or may be retained. The fluorescent marker may comprise an identifying feature, such as a nucleic acid barcode sequence and/or unique molecular identifier.

In another example, a first sample comprising a first plurality of cells including a first plurality of cells of a given type (e.g., immune cells expressing a particular marker or combination of markers) and a second sample comprising a second plurality of cells including a second plurality of cells of the given type may be subjected to a positive separation process. The first and second samples may be collected from the same or different subjects, at the same or different types, from the same or different bodily locations or systems, using the same or different collection techniques. For example, the first sample may be from a first subject and the second sample may be from a second subject different than the first subject. The first plurality of cells of the first sample may be provided a first plurality of fluorescent markers configured to label the first plurality of cells of the given type. The second plurality of cells of the second sample may be provided a second plurality of fluorescent markers configured to label the second plurality of cells of the given type. The first plurality of fluorescent markers may include a first identifying feature, such as a first barcode, while the second plurality of fluorescent markers may include a second identifying feature, such as a second barcode, that is different than the first identifying feature. The first plurality of fluorescent markers and the second plurality of fluorescent markers may fluoresce at the same intensities and over the same range of wavelengths upon excitation with a same excitation source (e.g., light source, such as a laser). The first and second samples may then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples may undergo separate FACS processes and the positively selected cells of the given type from the first sample and the positively selected cells of the given type from the second sample may then be combined for subsequent analysis. The encoded identifying features of the different fluorescent markers may be used to identify cells originating from the first sample and cells originating from the second sample. For example, the first and second identifying features may be configured to interact (e.g., in partitions, as described herein) with nucleic acid barcode molecules (e.g., as described herein) to generate barcoded nucleic acid products detectable using, e.g., nucleic acid sequencing.

### C. Beads

A partition may comprise one or more unique identifiers, such as barcodes (e.g., a plurality of barcode nucleic acid molecules which comprise a plurality of partition barcode sequences). Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof. Methods and systems for partitioning barcode carrying beads into droplets are provided in US. Pat. Publication Nos. 2019/0367997 and 2019/0064173, and International Application Nos. PCT/US20/17785 and PCT/US20/020486, each of which is herein entirely incorporated by reference for all purposes.

In some examples, beads, biological particles, and droplets may flow along channels (e.g., the channels of a microfluidic device). In some examples, beads, biological particles and droplets may flow along channels (e.g., the channels of a microfluidic device), in some cases at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Pat. Publication No. 2015/0292988, which is entirely incorporated herein by reference.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (µm), 5 µm, 10 µm, 20 µm, 30 µm, 40 µm, 50 µm, 60 µm, 70 µm, 80 µm, 90 µm, 100 µm, 250 µm, 500 µm, 1 mm, or greater. In some cases, a bead may have a diameter of less than about 10 nm, 100 nm, 500 nm, 1 µm, 5 µm, 10 µm, 20 µm, 30 µm, 40 µm, 50 µm, 60 µm, 70 µm, 80 µm, 90 µm, 100 µm, 250 µm, 500 µm, 1 mm, or less. In some cases, a bead may have a diameter in the range of about 40-75 µm, 30-75 µm, 20-75 µm, 40-85 µm, 40-95 µm, 20-100 µm, 10-100 µm, 1-100 µm, 20-250 µm, or 20-500 µm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Pat. Pub. Nos. 2014/0378345 and 2015/0376609, each of which is entirely incorporated herein by reference. The generation of a barcoded sequence, see, e.g., FIG. 3, is described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide, such as a barcoded nucleic acid molecule) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes (e.g., partition barcode sequences). A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached, e.g., bound, barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecular tag molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photosensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads (e.g., capture agent) during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about ⅒th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition (e.g., multi-omic method described elsewhere, herein). Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (µL), 5 µL, 1 µL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### D. Reagents

In accordance with certain aspects, biological particles may be partitioned along with lysis reagents in order to release the contents of the biological particles within the partition. See, e.g., U.S. Pat. Pub. 2018/0216162 (now U.S. Pat. 10,428,326), U.S. Pat. Pub. 2019/0100632 (now U.S. Pat. 10,590,244), and U.S. Pat. Pub. 2019/0233878, which are incorporated by reference in their entirety. Cell beads may be partitioned together with nucleic acid barcode molecules and the nucleic acid molecules of or derived from the cell bead (e.g., mRNA, cDNA, gDNA, secreted antibodies or antigen binding fragments thereof, etc.) can be barcoded as described elsewhere herein. In some embodiments, cell beads are co-partitioned with barcode carrying beads (e.g., gel beads) and the nucleic acid molecules of or derived from the cell bead are barcoded as described elsewhere herein. In such cases, the lysis agents can be contacted with the biological particle suspension concurrently with, or immediately prior to, the introduction of the biological particles into the partitioning junction/droplet generation zone, such as through an additional channel or channels upstream of the channel junction. In accordance with other aspects, additionally or alternatively, biological particles may be partitioned along with other reagents, as will be described further below.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structures may have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle’s contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle’s DNA, DNA polymerase enzymes and dNTPs used to amplify the biological particle’s nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, proteins, or secreted antibodies or antigen binding fragments thereof) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle’s macromolecular components (and as a result, its characteristics) with the unique identifiers. These unique identifiers can then be used to attribute the biological particle’s components and characteristics to an individual biological particle or group of biological particles.

In some aspects, this is performed by co-partitioning the individual biological particle or groups of biological particles with the unique identifiers, such as described above (with reference to FIGS. 1 and 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h₀, α, etc.) of the channel structure 200. A plurality of droplets can be collected in the reservoir 204 by continuously injecting the aqueous fluid 208 from the channel segment 202 through the junction 206.

A discrete droplet generated may include a bead (e.g., as in occupied droplets 216). Alternatively, a discrete droplet generated may include more than one bead. Alternatively, a discrete droplet generated may not include any beads (e.g., as in unoccupied droplet 218). In some instances, a discrete droplet generated may contain one or more biological particles, as described elsewhere herein. In some instances, a discrete droplet generated may comprise one or more reagents, as described elsewhere herein.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can comprise biological particles (e.g., described with reference to FIG. 1). In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles.

The second fluid 210 can comprise an oil, such as a fluorinated oil, that includes a fluorosurfactant for stabilizing the resulting droplets, for example, inhibiting subsequent coalescence of the resulting droplets.

In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets.

The channel structure 200 at or near the junction 206 may have certain geometric features that at least partly determine the sizes of the droplets formed by the channel structure 200. The channel segment 202 can have a height, h₀ and width, w, at or near the junction 206. By way of example, the channel segment 202 can comprise a rectangular cross-section that leads to a reservoir 204 having a wider cross-section (such as in width or diameter). Alternatively, the cross-section of the channel segment 202 can be other shapes, such as a circular shape, trapezoidal shape, polygonal shape, or any other shapes. The top and bottom walls of the reservoir 204 at or near the junction 206 can be inclined at an expansion angle, α. The expansion angle, α, allows the tongue (portion of the aqueous fluid 208 leaving channel segment 202 at junction 206 and entering the reservoir 204 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 \left(1 + 2.2 \sqrt{tan α} \frac{w}{{h}_{0}}\right) \frac{{h}_{0}}{\sqrt{tan α}}$$

By way of example, for a channel structure with w = 21 µm, h = 21 µm, and α = 3°, the predicted droplet size is 121 µm. In another example, for a channel structure with w = 25 µm, h = 25 µm, and α = 5°, the predicted droplet size is 123 µm. In another example, for a channel structure with w = 28 µm, h = 28 µm, and α = 7°, the predicted droplet size is 124 µm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (µm) to about 500 µm. In some instances, the width, w, can be between a range of from about 10 µm to about 200 µm. Alternatively, the width can be less than about 10 µm. Alternatively, the width can be greater than about 500 µm. In some instances, the flow rate of the aqueous fluid 208 entering the junction 206 can be between about 0.04 microliters (µL)/minute (min) and about 40 µL/min. In some instances, the flow rate of the aqueous fluid 208 entering the junction 206 can be between about 0.01 microliters (µL)/minute (min) and about 100 µL/min. Alternatively, the flow rate of the aqueous fluid 208 entering the junction 206 can be less than about 0.01 µL/min. Alternatively, the flow rate of the aqueous fluid 208 entering the junction 206 can be greater than about 40 µL/min, such as 45 µL/min, 50 µL/min, 55 µL/min, 60 µL/min, 65 µL/min, 70 µL/min, 75 µL/min, 80 µL/min, 85 µL/min, 90 µL/min, 95 µL/min, 100 µL/min, 110 µL/min, 120 µL/min, 130 µL/min, 140 µL/min, 150 µL/min, or greater. At lower flow rates, such as flow rates of about less than or equal to 10 microliters/minute, the droplet radius may not be dependent on the flow rate of the aqueous fluid 208 entering the junction 206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 206) between aqueous fluid 208 channel segments (e.g., channel segment 202) and the reservoir 204. Alternatively or in addition, the throughput of droplet generation can be increased by increasing the flow rate of the aqueous fluid 208 in the channel segment 202.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions comprising a barcode bead (e.g., a gel bead) associated with barcode molecules and a bead encapsulating cellular constituents (e.g., a cell bead) such as cellular nucleic acids can be useful in constituent analysis as is described in U.S. Pat. Publication No. 2018/0216162, which is herein incorporated by reference in its entirety for all purposes.

### E. Microwells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein. The wells or microwells may be initially provided in a “closed” or “sealed” configuration, wherein they are not accessible on a planar surface of the substrate without an external force. For instance, the “closed” or “sealed” configuration may comprise a substrate such as a sealing film or foil that is puncturable or pierceable by pipette tip(s). Suitable materials for the substrate include, without limitation, polyester, polypropylene, polyethylene, vinyl, and aluminum foil.

The well may have a volume of less than 1 milliliter (mL). For instance, the well may be configured to hold a volume of at most 1000 microliters (µL), at most 100 µL, at most 10 µL, at most 1 µL, at most 100 nanoliters (nL), at most 10 nL, at most 1 nL, at most 100 picoliters (pL), at most 10 (pL), or less. The well may be configured to hold a volume of about 1000 µL, about 100 µL, about 10 µL, about 1 µL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 µL, at least 10 µL, at least 100 µL, at least 1000 µL, or more. The well may be configured to hold a volume in a range of volumes listed herein, for example, from about 5 nL to about 20 nL, from about 1 nL to about 100 nL, from about 500 pL to about 100 µL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a microcapsule or bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the microcapsule or bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

A well may comprise free reagents and/or reagents encapsulated in, or otherwise coupled to or associated with, microcapsules, beads, or droplets. Any of the reagents described in this disclosure may be encapsulated in, or otherwise coupled to, a microcapsule, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, reporter barcode sequences, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well may be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a microcapsule, bead, or droplet that comprises a set of reagents that has a similar attribute (e.g., a set of enzymes, a set of minerals, a set of oligonucleotides, a mixture of different barcode molecules, a mixture of identical barcode molecules). In other cases, a microcapsule, bead, or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different microcapsule, droplet, or bead, or within a solution within a partition (e.g., microwell) of the system.

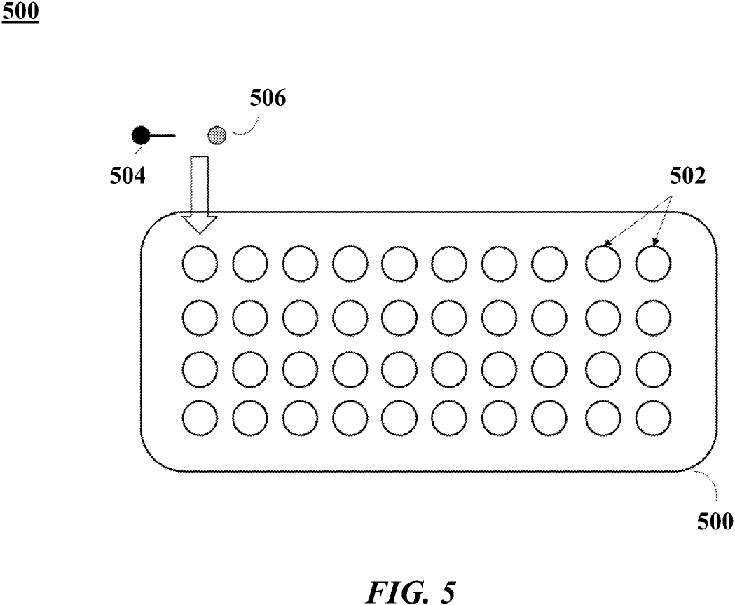

FIG. 5 schematically illustrates an example of a microwell array. The array can be contained within a substrate 500. The substrate 500 comprises a plurality of wells 502. The wells 1002 may be of any size or shape, and the spacing between the wells, the number of wells per substrate, as well as the density of the wells on the substrate 500 can be modified, depending on the particular application. In one such example application, a sample molecule 506, which may comprise a cell or cellular components (e.g., nucleic acid molecules) is co-partitioned with a bead 504, which may comprise a nucleic acid barcode molecule coupled thereto. The wells 502 may be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents may be loaded into a well either sequentially or concurrently. In some cases, reagents are introduced to the device either before or after a particular operation. In some cases, reagents (which may be provided, in certain instances, in microcapsules, droplets, or beads) are introduced sequentially such that different reactions or operations occur at different steps. The reagents (or microcapsules, droplets, or beads) may also be loaded at operations interspersed with a reaction or operation step. For example, microcapsules (or droplets or beads) comprising reagents for fragmenting polynucleotides (e.g., restriction enzymes) and/or other enzymes (e.g., transposases, ligases, polymerases, etc.) may be loaded into the well or plurality of wells, followed by loading of microcapsules, droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a microcapsule, bead, or droplet. These microcapsules, beads, or droplets may be loaded into a partition (e.g., a microwell) before, after, or concurrently with the loading of a cell, such that each cell is contacted with a different microcapsule, bead, or droplet. This technique may be used to attach a unique nucleic acid barcode molecule to nucleic acid molecules obtained from each cell. Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or microcapsule may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

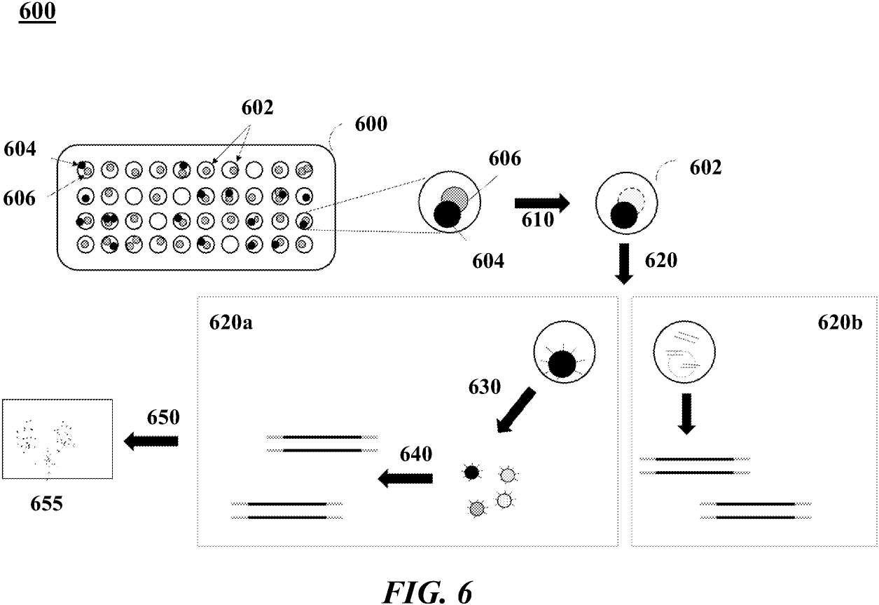

FIG. 6 schematically shows an example workflow for processing nucleic acid molecules within a sample. A substrate 600 comprising a plurality of microwells 602 may be provided. A sample 606 which may comprise a cell, cell bead, cellular components or analytes (e.g., proteins and/or nucleic acid molecules) can be co-partitioned, in a plurality of microwells 602, with a plurality of beads 604 comprising nucleic acid barcode molecules. During process 610, the sample 606 may be processed within the partition. For instance, in the case of live cells, the cell may be subjected to conditions sufficient to lyse the cells and release the analytes contained therein. In process 620, the bead 604 may be further processed. By way of example, processes 620a and 620b schematically illustrate different workflows, depending on the properties of the bead 604.

In 620a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 630, the beads 604 from multiple wells 602 may be collected and pooled. Further processing may be performed in process 640. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

In 620b, the bead comprises nucleic acid barcode molecules that are releasably attached thereto, as described below. The bead may degrade or otherwise release the nucleic acid barcode molecules into the well 602; the nucleic acid barcode molecules may then be used to barcode nucleic acid molecules within the well 602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

### VII. Detection and Analysis

### A. Generation and Analysis of Barcoded Molecules

Once the contents of the cells are released into their respective partitions, the nucleic acids contained therein may be further processed within the partitions. In accordance with the methods and systems described herein, the nucleic acid contents of individual cells can be provided with unique identifiers such that, upon characterization of those nucleic acids they may be attributed as having been derived from the same cell or cells. The ability to attribute characteristics to individual cells or groups of cells is provided by the assignment of unique identifiers specifically to an individual cell or groups of cells. Unique identifiers, e.g., in the form of nucleic acid barcodes can be assigned or associated with individual cells or populations of cells, in order to tag or label the cell’s components (and as a result, its characteristics) with the unique identifiers. These unique identifiers can then be used to attribute the cell’s components and characteristics to an individual cell or group of cells. In some aspects, this is carried out by co-partitioning the individual cells or groups of cells with the unique identifiers. In some aspects, the unique identifiers are provided in the form of oligonucleotides (also referred to herein as anchor oligonucleotides) that comprise nucleic acid barcode sequences that may be attached to or otherwise associated with the nucleic acid contents of individual cells, or to other components of the cells, and particularly to fragments of those nucleic acids. The oligonucleotides may be partitioned such that as between oligonucleotides in a given partition, the nucleic acid barcode sequences contained therein are the same, but as between different partitions, the oligonucleotides can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

In some aspects, the provided methods involve analyzing, e.g., detecting or determining, for imaging.

In some embodiments, disclosed herein is a method for screening an antigen, comprising:
(a) contacting an immune receptor with a plurality of engineered yeast cells to yield an engineered yeast cell bound to the immune receptor, wherein the plurality of engineered yeast cells comprise (i) an antigen comprising a polypeptide sequence; and (ii) a first nucleic acid molecule comprising a sequence encoding for the polypeptide sequence;
(b) generating a plurality of partitions, wherein a partition of the plurality of partitions comprises (i) the engineered yeast cell bound to the immune receptor; and (ii) a plurality of nucleic acid barcode molecules comprising a common barcode sequence;
(c) generating a second nucleic acid molecule comprising (i) a sequence corresponding to the polypeptide sequence and (ii) a sequence corresponding to the common barcode sequence.

In some embodiments, in (a), the antigen is displayed on the surface of the plurality of engineered yeast cells. In some embodiments, the antigen is attached, covalently or non-covalently (e.g., through a binding pair), to a yeast cell surface anchor protein. In some embodiments, the yeast cell surface anchor protein comprises a glycosylphosphatidylinositol (GPI) anchor. In some embodiments, the yeast cell surface anchor protein is Aga2p. In some embodiments, the antigen is part of a fusion protein, e.g., with the yeast cell surface anchor protein.

In some embodiments, the plurality of nucleic acid barcode molecules further comprise a capture sequence and wherein the first nucleic acid molecule further comprises a sequence configured to hybridize with the capture sequence. In some embodiments, (c) comprises hybridizing the first nucleic acid molecule to a nucleic acid barcode molecule of the plurality of nucleic acid barcode molecules and performing a nucleic acid extension reaction to generate the second nucleic acid molecule. In some embodiments, (c) comprises hybridizing the first nucleic acid molecule to a nucleic acid barcode molecule of the plurality of nucleic acid barcode molecules and performing a ligation reaction to generate the second nucleic acid molecule. In some embodiments, the method further comprises sequencing the first nucleic acid molecule or derivative thereof to generate sequencing reads corresponding to the polypeptide sequence of the antigen and the common barcode sequence.

In some embodiments, the immune receptor is a B cell receptor, an antibody, or an antigen binding fragment or derivative thereof. In some embodiments, (a) comprises contacting a cell comprising the immune receptor with the plurality of engineered yeast cells and wherein, in (b), the partition comprises the engineered yeast cell bound to the cell. In some embodiments, the cell is a B cell. In some embodiments, the cell comprises a messenger ribonucleic acid (mRNA) molecule encoding for the immune receptor and further comprising, following (b), generating a third nucleic acid molecule comprising (i) a sequence corresponding to the immune receptor and (ii) a sequence corresponding to the common barcode sequence. In some embodiments, the plurality of nucleic acid barcode molecules further comprise a capture sequence and wherein (c) comprises hybridizing the mRNA molecule or a fragment thereof to a nucleic acid barcode molecule of the plurality of nucleic acid barcode molecules and performing a nucleic acid extension reaction to generate the third nucleic acid molecule.

In some embodiments, the partition further comprises a fourth nucleic acid molecule comprising a poly-T sequence, wherein the plurality of nucleic acid barcode molecules further comprise a template switching oligonucleotide (TSO) sequence, and wherein (c) comprises (i) using the fourth nucleic acid molecule and the mRNA molecule to generate a complementary deoxyribonucleic acid (cDNA) molecule comprising the sequence corresponding to the immune receptor and (ii) performing a template switching reaction using a nucleic acid barcode molecule of the plurality of nucleic acid barcode molecules to generate the third nucleic acid molecule.

In some embodiments, the method further comprises sequencing the first nucleic acid molecule or derivative thereof to generate sequencing reads corresponding to the polypeptide sequence of the antigen and the common barcode sequence. In some embodiments, the method further comprises sequencing the third nucleic acid molecule or derivative thereof to generate sequencing reads corresponding to the immune receptor (e.g., B cell receptor, an antibody, or an antigen binding fragment or derivative thereof) and the common barcode sequence. In some embodiments, the method further comprises using the sequencing reads corresponding to the common barcode sequence to associate the immune receptor and the polypeptide sequence and/or the antigen comprising the polypeptide sequence.

In some embodiments, the plurality of nucleic acid barcode molecules is attached to a solid support. In some embodiments, the solid support is a bead. In some embodiments, the plurality of nucleic acid barcode molecules is releasably attached to the bead. In some embodiments, the method further comprises releasing the plurality of nucleic acid barcode molecules from the bead. In some embodiments, the bead is a gel bead. In some embodiments, the gel bead is a degradable upon application of a stimulus. In some embodiments, the stimulus is a chemical stimulus. In some embodiments, the partition comprises the chemical stimulus. In some embodiments, the plurality of partitions is a plurality of aqueous droplets in an emulsion. In some embodiments, the plurality of partitions is a plurality of wells.

Barcodes as described herein may be used to associate one or more analytes (e.g., secreted molecules or cellular nucleotide sequences such as mRNAs) with a cell and/or a partition (e.g., droplet or well) upon analyzing the barcodes using e.g., sequencing reads generated using an Illumina sequencer.

The herein described methods may comprise use of multiple barcodes to analyze multiple analytes and cellular molecules such as antibodies and/or mRNA molecules. A barcode may be a nucleic acid sequence (barcode sequence). A first barcode may be different from a second barcode. For example, the nucleic acid sequence of a first barcode sequence may be different from that of a second barcode sequence. As described herein, nucleic acid molecules comprising a barcode sequence may be coupled to other molecules, polymer, or particles. For example, nucleic acid molecules comprising a barcode sequence may be coupled to MHC molecules (e.g., tetrameric MHC-peptide complexes comprising a barcode sequence), secondary binding agents (e.g., an antibody coupled to a barcode sequence), polymers (e.g., dextramers or polymers capable of forming hydrogels), and/or beads (e.g., beads in emulsion droplets or in wells of a microwell array). Use of one or more barcodes may allow measurement and analysis of one or more analytes of a cell and may allow associating the one or more analytes with the respective cell. This may be particularly advantageous when measuring and analyzing multiple analytes (e.g., secreted molecules and/or mRNAs) from a single cell or from a plurality of cells such as one or more cell populations (e.g., immune cells). In such cases, a first barcode may be used to measure a first analyte of a cell, and a second barcode may be used to measure a second analyte of the cell (e.g., an mRNA molecule), and so forth. In these instances, a partition or cell specific barcode (e.g., attached to a bead, such as a gel bead) may be utilized to link the first barcode and the second barcode to attribute one or more analytes to a single cell. The analysis of an immune cell (e.g., a T cell, B cell, plasma cell, or dendritic cell), for example, may comprise measuring one or more signaling molecules (e.g., antibodies) that may be secreted upon stimulation of the cell (e.g., by using a stimulatory molecule), and one or more mRNA molecules that may be released from the cell upon cell lysis for analyzing, e.g., immune cell receptor gene segments (e.g., a V(D)J sequence of a T cell receptor (TCR)). See, e.g., U.S. Pat. Pub. 2018/0105808, which is incorporated by reference in its entirety, for exemplary molecules and methods for analyzing V(D)J sequences of single cells using nucleic acid barcode molecules.

FIG. 3 illustrates an example of a barcode carrying bead. A nucleic acid molecule 302, such as an oligonucleotide, can be coupled to a bead 304 by a releasable linkage 306, such as, for example, a disulfide linker. The same bead 304 may be coupled (e.g., via releasable linkage) to one or more other nucleic acid molecules 318, 320. The nucleic acid molecule 302 may be or comprise a barcode. As noted elsewhere herein, the structure of the barcode may comprise a number of sequence elements. The nucleic acid molecule 302 may comprise a functional sequence 308 that may be used in subsequent processing. For example, the functional sequence 308 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 302 may comprise a barcode sequence 310 for use in barcoding the sample (e.g., DNA, RNA, protein, antibody, etc.). In some cases, the barcode sequence 310 can be bead-specific such that the barcode sequence 310 is common to all nucleic acid molecules (e.g., including nucleic acid molecule 302) coupled to the same bead 304. Alternatively or in addition, the barcode sequence 310 can be partition-specific such that the barcode sequence 310 is common to all nucleic acid molecules coupled to one or more beads that are partitioned into the same partition. The nucleic acid molecule 302 may comprise a specific priming sequence 312, such as an mRNA specific priming sequence (e.g., poly-T sequence), a targeted priming sequence, and/or a random priming sequence. The nucleic acid molecule 302 may comprise an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 302 may comprise a unique molecular identifying sequence 316 (e.g., unique molecular identifier (UMI)). In some cases, the unique molecular identifying sequence 316 may comprise from about 5 to about 8 nucleotides. Alternatively, the unique molecular identifying sequence 316 may compress less than about 5 or more than about 8 nucleotides. The unique molecular identifying sequence 316 may be a unique sequence that varies across individual nucleic acid molecules (e.g., 302, 318, 320, etc.) coupled to a single bead (e.g., bead 304). In some cases, the unique molecular identifying sequence 316 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 3 shows three nucleic acid molecules 302, 318, 320 coupled to the surface of the bead 304, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 308, 310, 312, etc.) and variable or unique sequence segments (e.g., 316) between different individual nucleic acid molecules coupled to the same bead.

A biological particle (e.g., cell, DNA, RNA, etc.) can be co-partitioned along with a barcode bearing bead 304. The barcoded nucleic acid molecules 302, 318, 320 can be released from the bead 304 in the partition. By way of example, in the context of analyzing sample RNA, the poly-T segment (e.g., 312) of one of the released nucleic acid molecules (e.g., 302) can hybridize to the poly-A tail of a mRNA molecule. Reverse transcription may result in a cDNA transcript of the mRNA, but which transcript includes each of the sequence segments 308, 310, 316 of the nucleic acid molecule 302. Because the nucleic acid molecule 302 comprises an anchoring sequence 314, it will more likely hybridize to and prime reverse transcription at the sequence end of the poly-A tail of the mRNA. Within any given partition, all of the cDNA transcripts of the individual mRNA molecules may include a common barcode sequence segment 310. However, the transcripts made from the different mRNA molecules within a given partition may vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

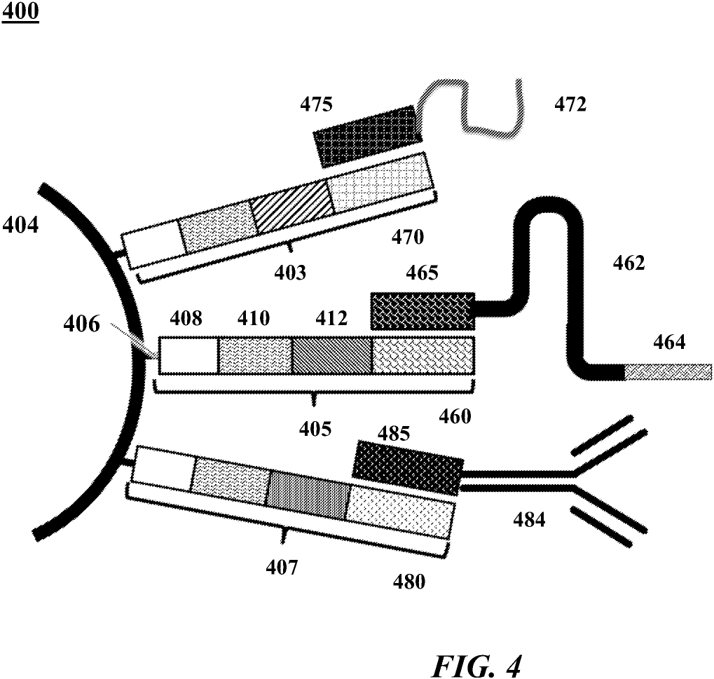

FIG. 4 illustrates another example of a barcode carrying bead. A nucleic acid molecule 405, such as an oligonucleotide, can be coupled to a bead 404 by a releasable linkage 406, such as, for example, a disulfide linker. The nucleic acid molecule 405 may comprise a first capture sequence 460. The same bead 404 may be coupled (e.g., via releasable linkage) to one or more other nucleic acid molecules 403, 407 comprising other capture sequences. The nucleic acid molecule 405 may be or comprise a barcode. As noted elsewhere herein, the structure of the barcode may comprise a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 may be configured to attach to a corresponding capture sequence 465. In some instances, the corresponding capture sequence 465 may be coupled to another molecule that may be an analyte or an intermediary carrier. For example, as illustrated in FIG. 4, the corresponding capture sequence 465 is coupled to a guide RNA molecule 462 comprising a target sequence 464, wherein the target sequence 464 is configured to attach to the analyte. Another oligonucleotide molecule 407 attached to the bead 404 comprises a second capture sequence 480 which is configured to attach to a second corresponding capture sequence 485. As illustrated in FIG. 4, the second corresponding capture sequence 485 is coupled to an antibody 482. In some cases, the antibody 482 may have binding specificity to an analyte (e.g., surface protein). Alternatively, the antibody 482 may not have binding specificity. Another oligonucleotide molecule 403 attached to the bead 404 comprises a third capture sequence 470 which is configured to attach to a second corresponding capture sequence 475. As illustrated in FIG. 4, the third corresponding capture sequence 475 is coupled to a molecule 472. The molecule 472 may or may not be configured to target an analyte. The other oligonucleotide molecules 403, 407 may comprise the other sequences (e.g., functional sequence, barcode sequence, UMI, etc.) described with respect to oligonucleotide molecule 405. While a single oligonucleotide molecule comprising each capture sequence is illustrated in FIG. 4, it will be appreciated that, for each capture sequence, the bead may comprise a set of one or more oligonucleotide molecules each comprising the capture sequence. For example, the bead may comprise any number of sets of one or more different capture sequences. Alternatively or in addition, the bead 404 may comprise other capture sequences. Alternatively or in addition, the bead 404 may comprise fewer types of capture sequences (e.g., two capture sequences). Alternatively or in addition, the bead 404 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

The herein disclosed methods and systems may further comprise partitioning cell beads into a plurality of partitions with a plurality of nucleic acid molecule comprising a cell-specific barcode sequence (see, e.g., the barcode molecules described in FIG. 3 or FIG. 4). In some instances, the cell specific barcodes are attached to a bead, such a gel bead. The cellular barcodes may be releasably attached to the bead as described elsewhere herein. Cell beads and cellular barcodes (e.g., attached to a bead, such as a gel bead) may be partitioned in a droplet or a well. The droplet may be an emulsion droplet and may comprise a cell bead. The emulsion droplet may be formed or generated as described elsewhere herein, e.g., by contacting two phases (e.g., a first and a second phase) that are immiscible (e.g., an aqueous phase and an oil). The cell bead may be dissolved using one or more stimuli such as change in pH, temperature, or ion concentration within the partition. In some instances, the cell is lysed, releasing cellular molecules such as nucleic acid molecules (e.g., mRNAs). The cell may be lysed prior to partitioning and barcoding of cellular molecules, e.g., analytes, or may be lysed in the partition. Cellular mRNA molecules may be reverse transcribed using nucleic acid molecules comprising a second barcode sequence, e.g., cell-specific barcode, thereby attaching the second barcode to the reversed transcribed nucleic acid molecules (and e.g., associating with other cellular molecules, e.g., analytes, of the cell).

Alternatively, cellular mRNA molecules may be first reverse transcribed into cDNA (e.g., using a poly-T containing primer) and the second, cell-specific barcode sequence attached (e.g., to the 5′ end of an mRNA/cDNA molecule) using, e.g., a template switching reaction as described elsewhere herein. See, e.g., U.S. Pat. Pub. 2018/0105808, which is incorporated by reference in its entirety, for exemplary molecules and methods for analyzing and barcoding mRNA of single cells using template switching reactions and template switching oligonucleotides. In some instances, cellular barcodes are released from, e.g., a bead (such as a gel bead) into the partition as described elsewhere herein (e.g., using a stimulus, such as a reducing agent). Similarly, the nucleic acid molecules comprising a first, analyte-specific barcode sequence can be utilized to generate a molecule comprising the first analyte specific barcode and the second, cell-specific barcode. The nucleic acid molecules attached to an analyte specific binding agent, e.g. reporter agent, may comprise one or more functional sequences in addition to the analyte-specific barcode sequence. For example, the nucleic acid molecules attached to an analyte specific binding agent may comprise one or more of a unique molecular identifier (UMI), a primer sequence or primer binding sequence (e.g., a sequencing primer sequence (or partial sequencing primer sequence) such as an R1 and/or R2 sequence), a sequence configured to attach to the flow cell of a sequencer (e.g., P5 and/or P7), or sequence complementary to a sequence on a nucleic acid barcode molecule (e.g., attached to a bead, such as those described in FIG. 3). Accordingly, barcoded molecules (e.g., comprising an analyte specific and cell specific barcode), may also comprise these functional sequences.

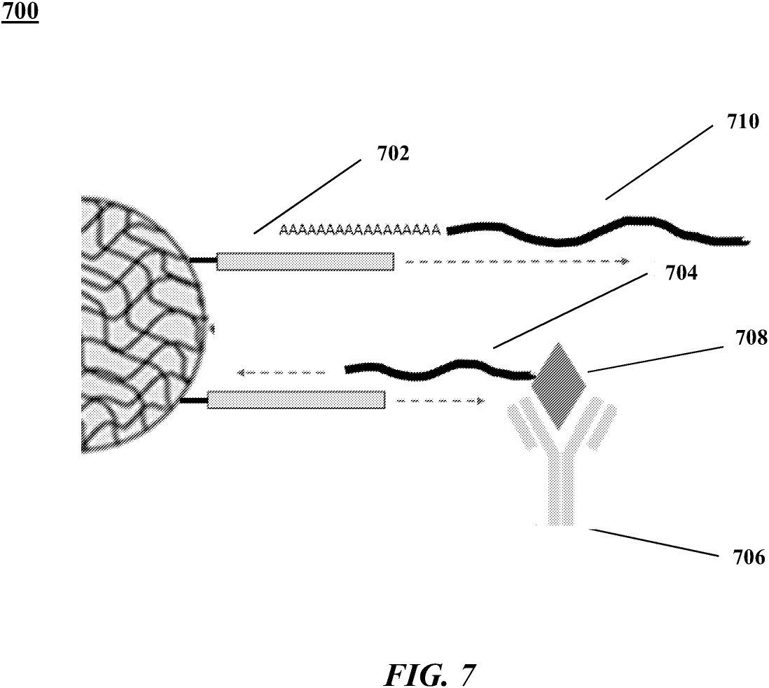

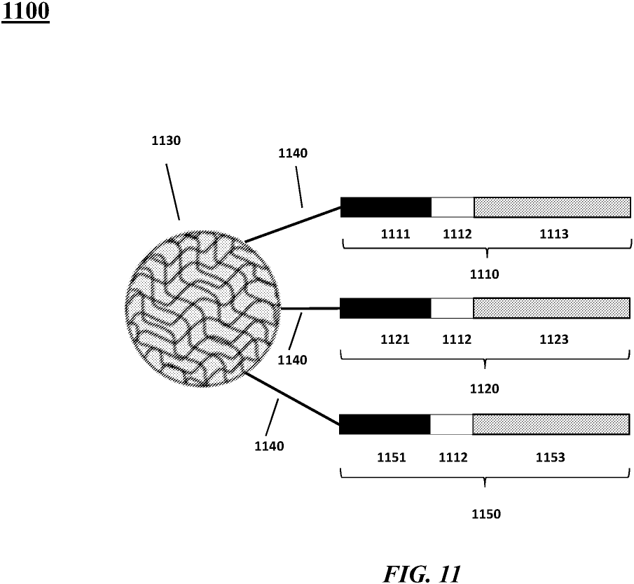

Once the contents of the cells are released into their respective partitions, the nucleic acids contained therein may be further processed within the partitions. In accordance with the methods and systems described herein, the nucleic acid contents of individual cells can be provided with unique identifiers such that, upon characterization of those nucleic acids they may be attributed as having been derived from the same cell or cells. The ability to attribute characteristics to individual cells or groups of cells is provided by the assignment of unique identifiers specifically to an individual cell or groups of cells. Unique identifiers, e.g., in the form of nucleic acid barcodes can be assigned or associated with individual cells or populations of cells, in order to tag or label the cell’s components (and as a result, its characteristics) with the unique identifiers. These unique identifiers can then be used to attribute the cell’s components and characteristics to an individual cell or group of cells. In some aspects, this is carried out by co-partitioning the individual cells or groups of cells with the unique identifiers. In some aspects, the unique identifiers are provided in the form of oligonucleotides (also referred to herein as anchor oligonucleotides) that comprise nucleic acid barcode sequences that may be attached to or otherwise associated with the nucleic acid contents of individual cells, or to other components of the cells, and particularly to fragments of those nucleic acids. The oligonucleotides may be partitioned such that as between oligonucleotides in a given partition, the nucleic acid barcode sequences contained therein are the same, but as between different partitions, the oligonucleotides can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present. FIG. 7 shows an example of a barcoded bead that may be used in a partition such as a droplet to couple a barcode 702 (e.g., a partition-specific barcode) and one or more analytes (e.g., an antigen or epitope expressed on a yeast cell surface and bound by an antibody in a serum or plasma sample or bound by a monoclonal antibody, mRNAs in the yeast cell including transcripts from an expression system expressing the antigen or epitope, etc.) of a single cell, thereby associating said one or more analytes with the single cell. For instance, partition-specific barcode 702 can be coupled to an mRNA 710 (which may be a transcript from the expression system introduced into a cell for expression of an antigen or epitope 708 or may comprise a barcode sequence corresponding to the antigen or epitope 708). Another molecule of the same partition-specific barcode may be coupled to reporter barcode 704 which corresponds to antigen or epitope 708 that binds to an antibody 706 that is present in a sample, such as a serum or plasma sample. FIG. 11 illustrates another example of a barcode carrying bead, as described in section VII-B.

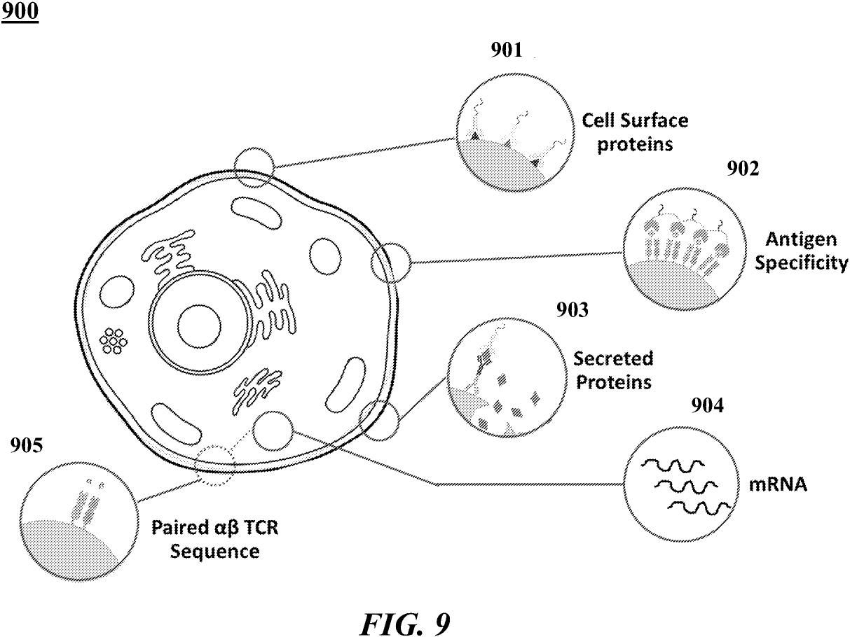

Upon completion of the one or more barcoding, reverse transcription, and/or nucleic acid processing steps (e.g., depending on how many different analytes of a cell are being barcoded), the contents of the partitions (e.g., droplets or wells) may be pooled and the nucleic acid molecules subjected to further bulk processing and sequencing. Thus, the presently described methods and systems allow the association of multiple analytes (e.g., secreted antibodies or antigen binding fragment thereof) to a single cell, thereby enabling the measurement, analysis, and/or characterization of a plurality of cells at the single cell level. As described herein, the plurality of cells (e.g., one or more cell populations such as populations of immune cells, e.g., B cells or plasma cells) may be analyzed and characterized in an efficient and simultaneous manner. The methods disclosed herein not only allow analysis of cellular molecules after lysis of the cell, but also allow analysis of molecules that may be secreted by the cell (e.g., an immune cell such as a T cell, B cell, plasma cell, or dendritic cell) such as secreted proteins, antibodies, antigen binding fragments thereof, or cytokines, (see e.g., operation 903) in addition and/or simultaneous to the analysis of cellular nucleic acid molecules (e.g., mRNAs for analyzing expression or presence of the exogenous epitope in an engineered cell, see e.g., operations 904), cell surface proteins (e.g., receptors or ligands, etc., see e.g., operation 901), antigen presentation (e.g., antigen presentation by B cells), antigen specificity (see e.g., operation 902) of a single cell, antigen receptor sequences (see e.g., operation 905), etc., e.g., as shown in FIG. 9.

In embodiments where intracellular analytes (e.g., mRNA) are processed in parallel, the cells contained in a partition (e.g., a droplet or a well) and cell beads contained in a partition (e.g., a droplet or a well), after cell beads are optionally dissolved (e.g., by dissolving the polymer matrix), are contacted with lysis reagents in order to release the contents of cells or viruses associated with the cell bead. In some cases, the lysis agents can be contacted with a cell bead suspension in bulk after cell bead formation. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), a surfactant based lysis solution (e.g., TritonX-100, Tween 20, sodium dodecyl sulfate (SDS)) for example, as well as other commercially available lysis enzymes. Electroporation, thermal, acoustic or mechanical cellular disruption may also be used in certain cases. In some cases, the cell bead matrix can be configured to give rise to a pore size that is sufficiently small to retain nucleic acid fragments of a particular size, following cellular disruption. In other instances, the cell bead matrix may be functionalized (e.g., covalently bound) with nucleic acid molecules (e.g., containing a poly-T sequence) configured to capture released analytes (e.g., mRNA, which optionally can be processed into cDNA prior to partitioning).

Other reagents can also be contacted with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cell beads, the cell beads may be exposed to an appropriate stimulus to release the cell beads or their contents into, e.g., a partition. For example, in some cases, a chemical stimulus may be co-partitioned along with an encapsulated cell bead to allow for the degradation of the microcapsule and release of the cell or its contents into the larger partition. In some cases, this stimulus may be the same as the stimulus described elsewhere herein for release of oligonucleotides from their respective microcapsule (e.g., bead). In alternative aspects, this may be a different and non-overlapping stimulus, in order to allow an encapsulated cell bead release its contents into a partition at a different time from the release of oligonucleotides into the same partition.

After the releasing of cellular macromolecular constituent, the cellular mRNA molecules are subject to a reverse transcription reaction with other types of reverse transcription primers such that cDNA is generated from the mRNAs. In some cases, simultaneously, a partition-specific (e.g., cell-specific, such as those described in FIG. 3) barcode molecule is attached during cDNA generation. In certain embodiments, the partition-specific (e.g., cell-specific) barcode sequence is a DNA oligonucleotide. The partition-specific (e.g., cell-specific) barcode sequence may be a second barcode sequence that is different from a first barcode sequence (e.g., analyte-specific barcode) attached to or coupled to a second binding reporter agent used to barcode an analyte that is secreted from a cell (e.g., an immune cell such as a B cell).

Alternatively, cellular mRNA molecules may be first reverse transcribed into cDNA (e.g., using a poly-T containing primer) and the second, cell-specific barcode sequence attached (e.g., to the 5′ end of an mRNA/cDNA molecule) using, e.g., a template switching reaction as described elsewhere herein. See, e.g., U.S. Pat. Pub. 2018/0105808, which is incorporated by reference in its entirety, for exemplary molecules and methods for analyzing and barcoding mRNA of single cells using template switching reactions and template switching oligonucleotides.

In certain embodiments, the partition-specific (e.g., cell-specific) barcode molecules (e.g., oligonucleotides, nucleic acid molecules) are releasable from the beads upon the application of a particular stimulus to the beads, as described elsewhere herein. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some embodiments, barcode molecules attached to reporter agent (e.g., an analyte specific polypeptide, such as an antigen) bound to an analyte may be released (e.g., through a releasable linkage/labile bound as described elsewhere herein). Similarly, in some embodiments, barcodes attached to MHC multimers and/or attached to cell surface protein specific antibodies may also be released (e.g., through a releasable linkage/labile bound as described elsewhere herein). Partition-specific (e.g., droplet-specific or secondary) barcode sequences may be attached to any or all of these released barcode molecules (or derivatives thereof). For example, a barcoded bead comprising a partition specific barcode sequence may be used in a partition such as a droplet to couple a barcode (e.g., a partition-specific barcode) to one or more analytes (e.g., secreted cytokines, mRNAs, etc) of a single cell, thereby associating said one or more analytes with the single cell. These barcodes are used as cell and/or partition-specific identifiers for RNA, DNA, proteins, secreted antibodies or antigen binding fragments thereof, and/or antigens that used to stimulate the cells. The assignment of unique barcodes specifically to an individual biological particle or groups of biological particles can attribute characteristics to individual biological particles or groups of biological particles. Unique identifiers, e.g., in the form of nucleic acid barcodes, are assigned or associated with individual biological particles or populations of biological particles, in order to tag or label the biological particle’s macromolecular components (and as a result, its characteristics) with the unique identifiers. These unique identifiers, e.g., barcodes, can then be used to attribute the biological particle’s components and characteristics to an individual biological particle or group of biological particles. Furthermore, as described elsewhere herein, in addition to cell and/or partition specific barcodes, unique molecular identifiers (UMIs) can also be added to cellular analytes (e.g., mRNA molecules) and reporter molecules (e.g., attached to binding agents, or reporter molecules attached to MHC molecules/multimers and/or antibodies, such as cell surface antibodies) to provide a unique identifier for quantitation of individual molecules.

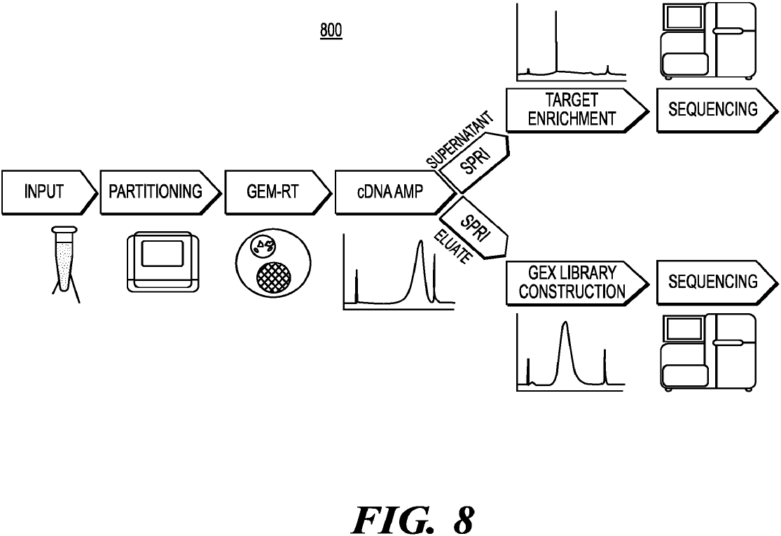

Barcoded partition contents are pooled into a bulk solution and further processed as described elsewhere herein to generate a sequencing library (see, e.g. FIG. 8). For example, following partitioning into Gel Bead-in-Emulsion (GEM), the cell in each droplet may be lysed and reverse transcription (GEM-RT) may be performed. After cDNA amplification, the supernatant and eluate are subjected to Solid Phase Reversible Immobilization (SPRI) followed by enrichment and library construction, respectively. Referring to FIG. 9, information regarding secreted antibodies, cytokines, as well as other analytes, such as mRNAs, cell surface proteins, and antigen binding specificity can all be analyzed and attributed to the same cell using the cell-specific barcode (see, e.g., FIG. 3 or FIG. 4).

### B. Multiplexing Methods

The present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells or cell features may be used to characterize cells and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In other aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more cells may be used to characterize a cell as belonging to a particular set of cells. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a cell may comprise subjecting the antibody linked to the oligonucleotide, e.g., antibody-linked barcode molecule, or the epitope binding fragment thereof linked to the reporter oligonucleotide, e.g., epitope binding fragment-linked barcode molecule to conditions suitable for binding the antibody to a molecule present on a surface of the cell. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 µM, 90 µM, 80 µM, 70 µM, 60 µM, 50 µM, 40 µM, 30 µM, 20 µM, 10 µM, 9 µM, 8 µM, 7 µM, 6 µM, 5 µM, 4 µM, 3 µM, 2 µM, 1 µM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 µM.

In another example, a reporter oligonucleotide may be coupled to a cell-penetrating peptide (CPP), and labeling cells may comprise delivering the CPP coupled to the reporter oligonucleotide, e.g., CPP coupled reporter oligonucleotide into an analyte carrier. Labeling analyte carriers may comprise delivering the CPP conjugated, e.g., coupled, oligonucleotide into a cell and/or cell bead by the cell-penetrating peptide. A CPP that can be used in the methods provided herein can comprise at least one non-functional cysteine residue, which may be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP may be an arginine-rich peptide transporter. The CPP may be Penetratin or the Tat peptide. In another example, a reporter oligonucleotide may be coupled to a fluorophore or dye, and labeling cells may comprise subjecting the fluorophore coupled to the reporter oligonucleotide, e.g., fluorophore-linked barcode molecule to conditions suitable for binding the fluorophore to the surface of the cell.

In some instances, fluorophores can interact strongly with lipid bilayers and labeling cells may comprise subjecting the fluorophore linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling cells may comprise delivering the reporter oligonucleotide, e.g., nucleic acid barcode molecule, to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane may be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus. In one embodiment, a reporter oligonucleotide coupled to a lipophilic molecule will remain associated with and/or inserted into lipid membrane (as described herein) via the lipophilic molecule until lysis of the cell occurs, e.g., inside a partition.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular reporter oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular cell population, or sample. Cell populations may be incubated with a plurality of libraries such that a cell or cells comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abeam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

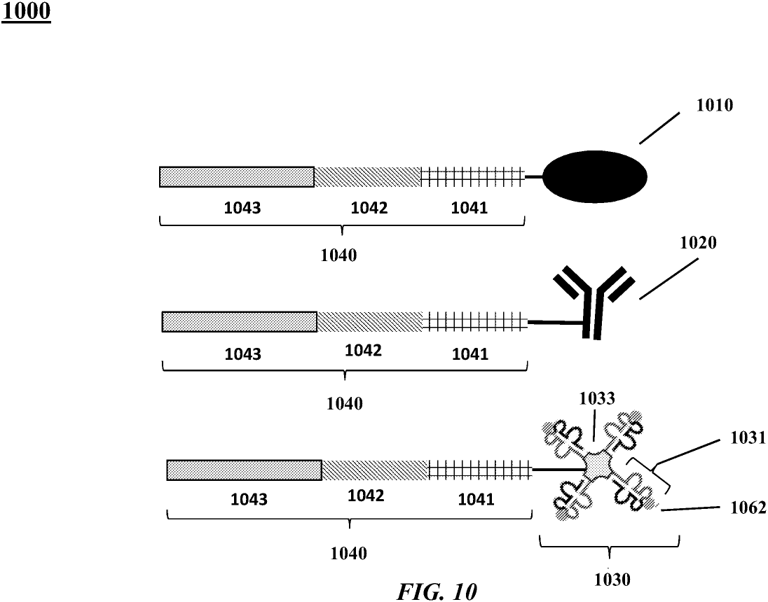

FIG. 10 describes exemplary labelling agents (1010, 1020, 1030) comprising reporter oligonucleotides (1040) attached thereto. Labelling agent 1010 (e.g., any of the labelling agents described herein) is attached (either directly, e.g., covalently attached, or indirectly) to reporter oligonucleotide 1040. Reporter oligonucleotide 1040 may comprise barcode sequence 1042 that identifies labelling agent 1010. Reporter oligonucleotide 1040 may also comprise one or more functional sequences 1043 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 10, in some instances, reporter oligonucleotide 1040 conjugated to a labelling agent (e.g., 1010, 1020, 1030) comprises a primer sequence 1041, a barcode sequence 1042 that identifies the labelling agent (e.g., 1010, 1020, 1030), and functional sequence 1043. Functional sequence 1043 may be configured to hybridize to a complementary sequence, such as a complementary sequence present on a nucleic acid barcode molecule 1090 (not shown), such as those described elsewhere herein. In some instances, nucleic acid barcode molecule 1090 is attached to a support (e.g., a bead, such as a gel bead), such as those described elsewhere herein. For example, nucleic acid barcode molecule 1090 may be attached to the support via a releasable linkage (e.g., comprising a labile bond), such as those described elsewhere herein. In some instances, reporter oligonucleotide 1040 comprises one or more additional functional sequences, such as those described above.

In some instances, the labelling agent 1010 is a protein or polypeptide (e.g., an antigen or prospective antigen) comprising reporter oligonucleotide 1040. Reporter oligonucleotide 1040 comprises barcode sequence 1042 that identifies polypeptide 1010 and can be used to infer the presence of an analyte, e.g., a binding partner of polypeptide 1010 (i.e., a molecule or compound to which polypeptide 1010 can bind). In some instances, the labelling agent 1010 is a lipophilic moiety (e.g., cholesterol) comprising reporter oligonucleotide 1040, where the lipophilic moiety is selected such that labelling agent 1010 integrates into a membrane of a cell or nucleus. Reporter oligonucleotide 1040 comprises barcode sequence 1042 that identifies lipophilic moiety 1010 which in some instances is used to tag cells (e.g., groups of cells, cell samples, etc.) and may be used for multiplex analyses as described elsewhere herein. In some instances, the labelling agent is an antibody 1020 (or an epitope binding fragment thereof) comprising reporter oligonucleotide 1040. Reporter oligonucleotide 1040 comprises barcode sequence 1042 that identifies antibody 1020 and can be used to infer the presence of, e.g., a target of antibody 1020 (i.e., a molecule or compound to which antibody 1020 binds). In other embodiments, labelling agent 1030 comprises an MHC molecule 1031 comprising peptide 1032 and reporter oligonucleotide 1040 that identifies peptide 1032. In some instances, the MHC molecule is coupled to a support 1033. In some instances, support 1033 may be a polypeptide, such as streptavidin, or a polysaccharide, such as dextran. In some instances, reporter oligonucleotide 1040 may be directly or indirectly coupled to MHC labelling agent 1030 in any suitable manner. For example, reporter oligonucleotide 1040 may be coupled to MHC molecule 1031, support 1033, or peptide 1032. In some embodiments, labelling agent 1030 comprises a plurality of MHC molecules, (e.g. is an MHC multimer, which may be coupled to a support (e.g., 1033)). There are many possible configurations of Class I and/or Class II MHC multimers that can be utilized with the compositions, methods, and systems disclosed herein, e.g., MHC tetramers, MHC pentamers MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. 10,550,429 and U.S. Pat. Pub. 20190367969, each of which is herein entirely incorporated by reference for all purposes.

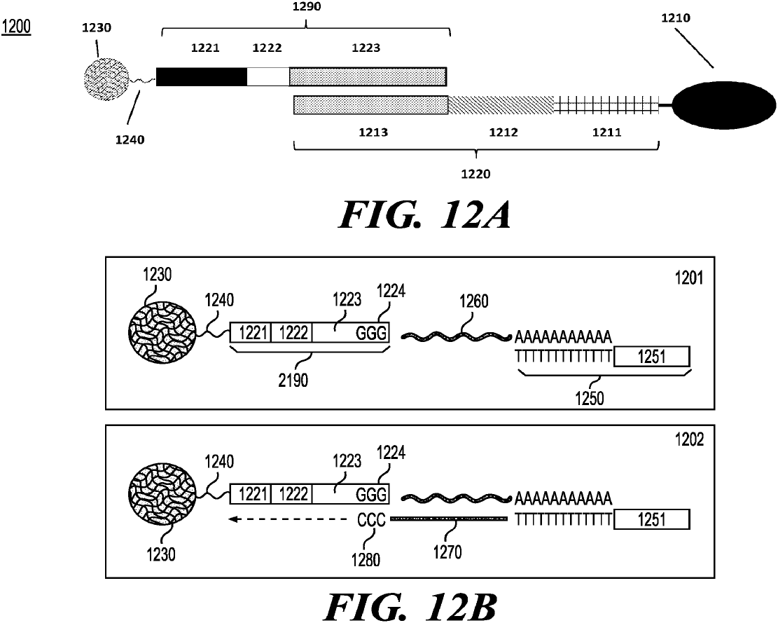

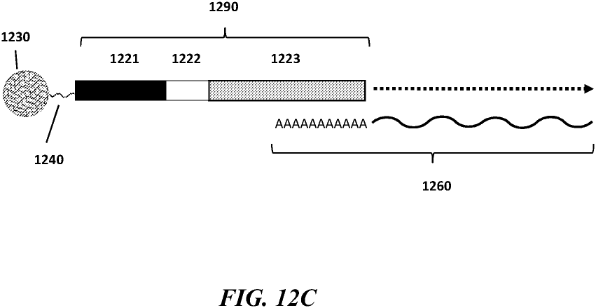

FIG. 11 illustrates another example of a barcode carrying bead. In some embodiments, analysis of multiple analytes (e.g., RNA and one or more analytes using labelling agents described herein) may comprise utilizing nucleic acid barcode molecules as generally depicted in FIG. 11. In some embodiments, nucleic acid barcode molecules 1110 and 1120 are attached to support 1130 via a releasable linkage 1140 (e.g., comprising a labile bond) as described elsewhere herein. Nucleic acid barcode molecule 1110 may comprise adapter sequence 1111, barcode sequence 1112 and adapter sequence 1113. Nucleic acid barcode molecule 1120 may comprise adapter sequence 1121, barcode sequence 1112, and adapter sequence 1123, wherein adapter sequence 1123 comprises a different sequence than adapter sequence 1113. In some instances, adapter 1111 and adapter 1121 comprise the same sequence. In some instances, adapter 1111 and adapter 1121 comprise different sequences. Although support 1130 is shown comprising nucleic acid barcode molecules 1110 and 1120, any suitable number of barcode molecules comprising common barcode sequence 1112 are contemplated herein. For example, in some embodiments, support 1130 further comprises nucleic acid barcode molecule 1150. Nucleic acid barcode molecule 1150 may comprise adapter sequence 1151, barcode sequence 1112 and adapter sequence 1153, wherein adapter sequence 1153 comprises a different sequence than adapter sequence 1113 and 1123. In some instances, nucleic acid barcode molecules (e.g., 1110, 1120, 1150) comprise one or more additional functional sequences, such as a UMI or other sequences described herein. The nucleic acid barcode molecules 1110, 1120 or 1150 may interact with analytes as described elsewhere herein, for example, as depicted in FIGS. 12A-C.

Referring to FIG. 12A, in an instance where cells are labelled with labeling agents, capture sequence 1223 may be complementary to a capture handle sequence of a reporter oligonucleotide. Cells may be contacted with one or more reporter oligonucleotide 1220 conjugated labelling agents 1210 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 1210 which is conjugated to oligonucleotide 1220 and support 1230 (e.g., a bead, such as a gel bead) comprising nucleic acid barcode molecule 1290 is partitioned into a partition amongst a plurality of partitions (e.g., a droplet of a droplet emulsion or a well of a microwell array). In some instances, the partition comprises at most a single cell bound to labelling agent 1210. In some instances, reporter oligonucleotide 1220 conjugated to labelling agent 1210 (e.g., polypeptide, an antibody, pMHC molecule such as an MHC multimer, etc.) comprises a first adapter sequence 1211 (e.g., a primer sequence), a barcode sequence 1212 that identifies the labelling agent 1210 (e.g., the polypeptide, antibody, or peptide of a pMHC molecule or complex), and a capture handle sequence 1213. Capture handle sequence 1213 may be configured to hybridize to a complementary sequence, such as a capture sequence 1223 present on a nucleic acid barcode molecule 1290. In some instances, oligonucleotide 1220 comprises one or more additional functional sequences, such as those described elsewhere herein.

Barcoded nucleic acid molecules may be generated (e.g., via a nucleic acid reaction, such as nucleic acid extension or ligation) from the constructs described in FIGS. 12A-C. For example, sequence 1213 may then be hybridized to complementary sequence 1223 to generate (e.g., via a nucleic acid reaction, such as nucleic acid extension or ligation) a barcoded nucleic acid molecule comprising cell (e.g., partition specific) barcode sequence 1222 (or a reverse complement thereof) and reporter sequence 1212 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 12A-C, or a combination of workflows for an individual analyte, as described elsewhere herein. For example, by using a combination of the workflows as generally depicted in FIGS. 12A-C, multiple analytes can be analyzed.

In some instances, analysis of an analyte (e.g. a nucleic acid, a polypeptide, a carbohydrate, a lipid, etc.) comprises a workflow as generally depicted in FIG. 12A. A nucleic acid barcode molecule 1290 may be co-partitioned with the one or more analytes. In some instances, nucleic acid barcode molecule 1290 is attached to a support 1230 (e.g., a bead, such as a gel bead), such as those described elsewhere herein. For example, nucleic acid barcode molecule 1290 may be attached to support 1230 via a releasable linkage 1240 (e.g., comprising a labile bond), such as those described elsewhere herein. Nucleic acid barcode molecule 1290 may comprise a barcode sequence 1221 and optionally comprise other additional sequences, for example, a UMI sequence 1222 (or other functional sequences described elsewhere herein). The nucleic acid barcode molecule 1290 may comprise a sequence 1223 that may be complementary to another nucleic acid sequence, such that it may hybridize to a particular sequence.

For example, sequence 1223 may comprise a poly-T sequence and may be used to hybridize to mRNA. Referring to FIG. 12C, in some embodiments, nucleic acid barcode molecule 1290 comprises sequence 1223 complementary to a sequence of RNA molecule 1260 from a cell. In some instances, sequence 1223 comprises a sequence specific for an RNA molecule. Sequence 1223 may comprise a known or targeted sequence or a random sequence. In some instances, a nucleic acid extension reaction may be performed, thereby generating a barcoded nucleic acid product comprising sequence 1223, the functional sequence 1221, common barcode sequence 1222, any other functional sequence, and a sequence corresponding to the RNA molecule 1260.

In another example, sequence 1223 may be complementary to an overhang sequence or an adapter sequence that has been appended to an analyte. For example, referring to FIG. 12B, in some embodiments, primer 1250 comprises a sequence complementary to a sequence of nucleic acid molecule 1260 (such as an RNA encoding for a BCR sequence) from an analyte carrier. In some instances, primer 1250 comprises one or more sequences 1251 that are not complementary to RNA molecule 1260. Sequence 1251 may be a functional sequence as described elsewhere herein, for example, an adapter sequence, a sequencing primer sequence, or a sequence the facilitates coupling to a flow cell of a sequencer. In some instances, primer 1250 comprises a poly-T sequence. In some instances, primer 1250 comprises a sequence complementary to a target sequence in an RNA molecule. In some instances, primer 1250 comprises a sequence complementary to a region of an immune molecule, such as the constant region of a TCR or BCR sequence. Primer 1250 is hybridized to nucleic acid molecule 1260 and complementary molecule 1270 is generated. For example, complementary molecule 1270 may be cDNA generated in a reverse transcription reaction. In some instances, an additional sequence may be appended to complementary molecule 1270. For example, the reverse transcriptase enzyme may be selected such that several non-templated bases 1280 (e.g., a poly-C sequence) are appended to the cDNA. In another example, a terminal transfer as may also be used to append the additional sequence. Nucleic acid barcode molecule 1290 comprises a sequence 1224 complementary to the non-templated bases, and the reverse transcriptase performs a template switching reaction onto nucleic acid barcode molecule 1290 to generate a barcoded nucleic acid molecule comprising cell (e.g., partition specific) barcode sequence 1222 (or a reverse complement thereof) and a sequence of complementary molecule 1270 (or a portion thereof). In some instances, sequence 1223 comprises a sequence complementary to a region of an immune molecule, such as the constant region of a TCR or BCR sequence. Sequence 1223 is hybridized to nucleic acid molecule 1260 and a complementary molecule 1270 is generated. For example, complementary molecule 1270 may be generated in a reverse transcription reaction generating a barcoded nucleic acid molecule comprising cell (e.g., partition specific) barcode sequence 1222 (or a reverse complement thereof) and a sequence of complementary molecule 1270 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Pat. Publication No. 2018/0105808, U.S. Pat. Publication No. 2015/0376609, filed Jun. 26, 2015, and U.S. Pat. Publication No. 2019/0367969, each of which applications is herein entirely incorporated by reference for all purposes.

### C. Computer Systems

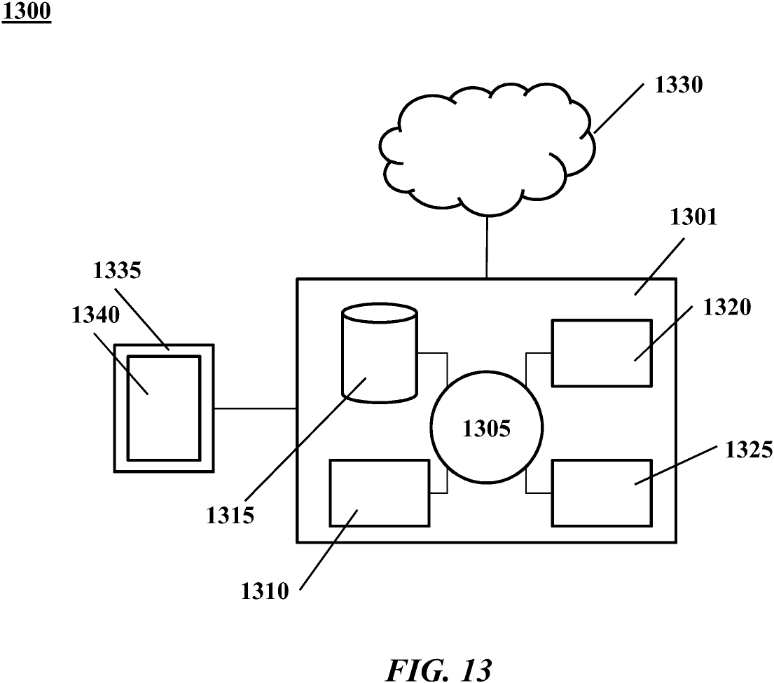

The present disclosure provides computer systems that are programmed to implement methods of the disclosure. FIG. 13 shows a computer system 1301 that is programmed or otherwise configured to (i) control a microfluidics system (e.g., fluid flow), (ii) sort occupied droplets from unoccupied droplets, (iii) polymerize droplets, (iv) partition cell beads or cells into partitions (e.g., droplets or wells), (v) lyse cells and cell beads, (vi) perform sequencing applications, (vii) generate and maintain libraries of cytokine or other analyte specific antibody barcode sequences, MHC multimer barcode sequences, cell surface protein barcode sequences, and cDNAs generated from mRNAs respectively (vi) analyze such libraries. The computer system 1301 can regulate various aspects of the present disclosure, such as, for example, regulating fluid flow rate in one or more channels in a microfluidic structure, regulating polymerization application units, regulating sequence application unit, etc. The computer system 1301 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user (e.g., operator). Examples of remote computer systems include personal computers (e.g., portable PC), slate or tablet PC’s (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1305. The algorithm can, for example, perform nucleotide sequence amplification, sequencing sorting based on barcode sizes, sequencing amplified barcode sequences, analyzing sequencing data, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

### VIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Determining Antigen-Specificity of Serum or Plasma Sample Using a Barcoded Yeast Cell Antigen Display System

Yeast cells are engineered to each uniquely display a single human, viral, bacterial, or other protein antigen of interest, to form a yeast cell library. In one exemplary yeast cell library, a plurality of yeast cells may each carry one of multiple variants of the same antigen (e.g. 80 variants of the same protein) to assess antigen-specificity of a serum and/or plasma profile. Specifically, a nucleic acid encoding the protein antigen of interest is cloned into a constitutively expressed site. The constitutively expressed site may further include a capture sequence designed for capture using other chemical methods. In addition, a first barcode sequence (or a nucleotide encoding a first barcode sequence) is also cloned into the genome of the yeast cell. The barcode may be uniquely corresponding to the antigen.

Peripheral blood is extracted from a subject, and plasma and/or serum are isolated from the collected, e.g., extracted, blood. The plasma and/or serum are placed in contact with the yeast cell library under a sufficient condition, wherein one or more of the yeast cells will be coated with antibodies specific for the unique antigen displayed. The yeast cells coated by antibodies may be enriched using magnetic, buoyancy, or other selection methods targeting the Fc region of the antibody.

After washing off the excess plasma and/or serum sample, yeast cells coated by antibodies can be partitioned into emulsion droplets and partitions may comprise beads that comprise a barcode that is different from the barcodes identifying the yeast cell displaying the unique antigen of interest. Cells may be lysed inside the partitions to release intracellular contents and two different types of barcodes. DNA amplifications and sequencing of the barcodes may be performed for identification of the displayed antigens and other intracellular analytes. As a result, the antigen-specificity of antibodies in the plasma and/or serum sample may be identified and quantified.

### Example 2: Determining Antigen-Specificity of Serum or Plasma Sample Using a Barcoded Yeast Cell Antigen Display System

Yeast cells are engineered to each uniquely display a single human, viral, bacterial, or other protein antigen of interest, to form a yeast cell library. Specifically, the nucleic acid encoding the protein antigen of interest is cloned into a constitutively expressed site. The constitutively expressed site may further include a capture sequence designed for capture using other chemical methods. In addition, a first barcode sequence (or a nucleotide encoding a first barcode sequence) is also cloned into the genome of the yeast cell. The barcode sequence may be uniquely corresponding to the antigen, e.g., antigen of interest. Additionally, a pool of yeast cells engineered to each uniquely express an antigen is further modified with knockdown against the antigen using a barcoding-detectable PERTURB-seq guide. This guide could also be attached to a molecule known to be internalized by the target-expressing cells. Therefore 2 pools of yeast cells are generated (antigen⁺ guide⁻ and antigen⁺ guide⁺)

Peripheral blood is extracted from a subject, and plasma and/or serum are isolated from the collected blood. The plasma and/or serum are placed in contact with the two pools of yeast cells under a sufficient condition, wherein one or more of the yeast cells will be coated with antibodies specific for the unique antigen displayed. The yeast cells coated by antibodies may be enriched using magnetic, buoyancy, or other selection methods targeting the Fc region of the antibody.

After washing off the excess plasma and/or serum sample, yeast cells coated by antibodies are partitioned into emulsion droplets and partitions may comprise beads that comprise a barcode that is different from the barcodes identifying the yeast cell displaying the unique antigen of interest and, optionally, the barcode corresponding to knockdown. Cells may be lysed inside the partitions to release intracellular contents and the different types of barcodes. DNA amplifications and sequencing of the barcodes may be performed for identification of the displayed antigens and other intracellular analytes. As a result, the antigen-specificity of antibodies in the plasma and/or serum sample may be identified and quantified.

For further control, the 2 population of yeast cells (antigen⁺ guide⁻ and antigen⁺ guide⁺) can be analyzed by using markers specific to each of the population to ensure equal representation for downstream analysis.

### Example 3: Determining Antigen-Specificity of Serum or Plasma Sample Using a Barcoded Yeast Cell Antigen Display System

Yeast cells are engineered to each uniquely display a single human, viral, bacterial, or other protein antigen of interest, to form a yeast cell library. Specifically, the nucleic acid encoding the protein antigen of interest is cloned into a constitutively expressed site. In addition, a first barcode sequence (or a nucleotide encoding a first barcode sequence) is also cloned into the genome of the yeast cell. The barcode may be uniquely corresponding to the antigen.

Peripheral blood is extracted from a subject, and plasma and/or serum are isolated from the collected, extracted, blood. The plasma and/or serum are placed in contact with the yeast cell library under a sufficient condition, wherein one or more of the yeast cells will be coated with antibodies specific for the unique antigen displayed. The yeast cells coated by antibodies may be enriched using magnetic, buoyancy, or other selection methods targeting the Fc region of the antibody.

After washing off the excess plasma and/or serum sample, yeast cells coated by antibodies are partitioned into microwells or microarrays such that each partition, to a high confidence level, carries no more than one yeast cell displaying an unique antigen of interest. Cells may be lysed inside the partitions to release intracellular contents and barcodes. DNA amplifications and sequencing of the barcodes may be performed for identification of the displayed antigens and other intracellular analytes. As a result, the antigen-specificity of antibodies in the plasma and/or serum sample may be identified and quantified.

### Example 4: Determining Antigen-Specificity of Serum or Plasma Sample Using a Barcoded Yeast Cell Antigen Display System

Yeast cells are engineered to each uniquely display a single human, viral, bacterial, or other protein antigen of interest, to form a yeast cell library. Specifically, the nucleic acid encoding the protein antigen of interest is cloned into a constitutively expressed site. The antigen is further engineered to contain a hexahistidine tag or other protein tags. The constitutively expressed site may further include a capture sequence designed for capture by other chemical methods. In addition, a first barcode sequence (or a nucleotide encoding a first barcode sequence) is also cloned into the genome of the yeast cell. The barcode may be uniquely corresponding to the antigen.

Peripheral blood is extracted from a subject, and plasma and/or serum are isolated from the collected, e.g., extracted, blood. The plasma and/or serum are placed in contact with the yeast cell library under a sufficient condition, wherein one or more of the yeast cells will be coated with antibodies specific for the unique antigen displayed. The yeast cells coated by antibodies may be enriched using magnetic, buoyancy, or other selection methods targeting the Fc region of the antibody.

The hexahistidine tag in specific antigens can be used for rapid oligonucleotide conjugation to generate a barcoded antigen. The hexahistidine tag can also be used for easy purification (such as by anti-His antibodies). These features will allow isolation of specific antibodies recognizing the specific antigen.

Optionally, after washing off the excess plasma and/or serum sample, yeast cells coated by antibodies can also be partitioned into emulsion droplets and partitions may comprise beads that comprise a barcode that is different from the barcodes identifying the yeast cell displaying the unique antigen of interest. Cells may be lysed inside the partitions to release intracellular contents and two different types of barcodes. DNA amplifications and sequencing of the barcodes may be performed for identification of the displayed antigens and other intracellular analytes. As a result, the antigen-specificity of antibodies in the plasma and/or serum sample may be identified and quantified.

### Example 5: Epitope Mapping for Monoclonal Antibodies Using a Barcoded Yeast Cell Antigen Display System

Yeast cells are engineered to each uniquely display an epitope from an antigen of interest. The epitopes may be partially overlapping or non-overlapping. For fine mapping of the antigen specificity, epitopes from an epitope variant panel may also be employed.

Specifically, the nucleic acid encoding the epitope of interest is cloned into a constitutively expressed site. The constitutively expressed site may further include a capture sequence designed for capture using other chemical methods. In addition, a first barcode sequence or a nucleotide encoding a first barcode sequence is also cloned into the genome of the yeast cell. The barcode may be uniquely corresponding to the epitope displayed.

One or more monoclonal antibodies will be tested against the yeast library of epitopes. A reporter barcode label must first be attached to the monoclonal antibody (mAb) of interest in order to confirm its binding specificity for one or more partners. This oligonucleotide should be unique to the candidate mAb of interest. This can be done directly or indirectly, and via multiple chemical methods varying in their site specificity. The monoclonal antibody could be engineered or fused to multiple different protein constructs to enable this labeling.

The barcoded mAbs are placed in contact with the yeast cell library under a sufficient condition, wherein one or more of the yeast cells will be coated with antibodies specific for the unique epitope displayed. The yeast cells coated by antibodies may be enriched using magnetic, buoyancy, or other selection methods targeting the Fc region of the antibody.

Yeast cells coated by reporter barcode labeled monoclonal antibodies are partitioned into emulsion droplets and partitions may comprise beads that comprise a barcode that is different from the barcodes identifying the yeast cell displaying the unique epitope of interest. Cells may be lysed inside the partitions to release intracellular contents and the two different types of barcodes. DNA amplifications and sequencing of the barcodes may be performed for identification of the displayed antigens and other intracellular analytes. As a result, the specificity of the reporter barcode labeled monoclonal antibody to the yeast epitope library may be identified and quantified, and correspondingly epitope mapping of the antibody can be analyzed.

### Example 6: Determining Antigen-Specificity and Epitope Mapping Using a Barcoded Yeast Cell Antigen Display and Antibody Secretion System

Yeast cells are engineered to express a monoclonal antibody of interest. The monoclonal antibody is converted to an IgG format and contains the exons for the constant regions for proper antibody expression. Furthermore, the antibody of interest is engineered to contain a modified N-terminal LPXTG motif.

The same yeast cells are further engineered to each uniquely display a target (either an epitope from an antigen of interest, or a variant of a protein antigen), thereby creating a yeast cell library for epitopes or for the variants. Specifically, the nucleic acid encoding the target is cloned into a constitutively expressed site. The target protein is further engineered to be fused with sortase A. In addition, a first barcode (or a nucleotide encoding a first barcode) can also be cloned into the genome of the yeast cell. The barcode may be uniquely corresponding to the epitope or the protein variant displayed.

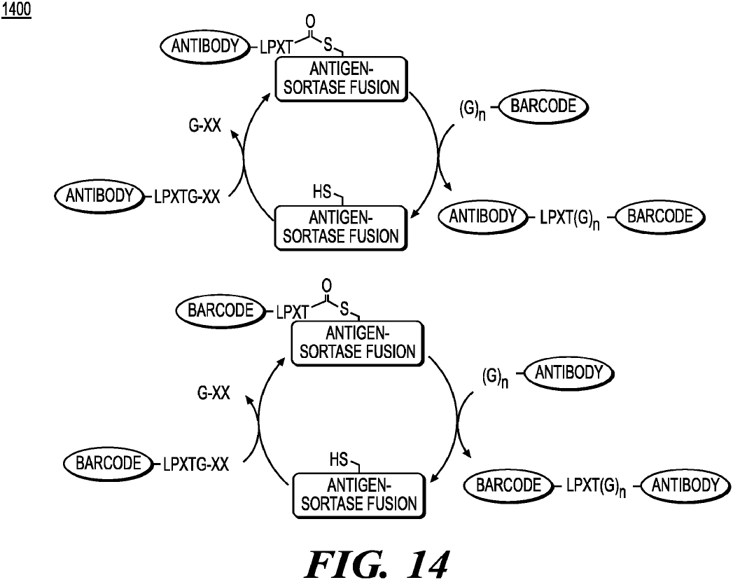

The yeast cells are placed under conditions for the monoclonal antibodies to be secreted and to bind to target displayed on the yeast cell. Upon secretion and binding of the secreted antibody to the target in the presence of polyglycine, the sortase A on the target protein will cleave the LPXTG motif on the antibody and replace the terminal glycine with polyglycine (G)ₙ, e.g., as shown in FIG. 14.

Optionally, after washing off the secreted antibodies, yeast cells coated by antibodies are partitioned into emulsion droplets and partitions may comprise beads that comprise a barcode that is different from the barcodes identifying the yeast cell displaying the unique epitope of interest. Cells may be lysed inside the partitions to release intracellular contents and the different types of barcodes. DNA amplifications and sequencing of the barcodes may be performed for identification of the displayed antigens and other intracellular analytes. As a result, the specificity of the barcode labeled monoclonal antibody to the yeast epitope or variant library may be identified and quantified, and correspondingly epitope mapping and/or antigen specificity of the antibody can be analyzed.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for analyzing a sample, comprising:
contacting a sample with a population of cells,
wherein the sample comprises a plurality of antibody molecules or antigen-binding fragments thereof,
wherein the population comprises: (i) a first cell that is engineered or otherwise modified to express a first antigen or epitope and that comprises a nucleic acid molecule comprising a first nucleic acid sequence (e.g., a first reporter barcode sequence) corresponding to the first antigen or epitope, and (ii) a second cell that is engineered or otherwise modified to express a second antigen or epitope and that comprises a nucleic acid molecule comprising a second nucleic acid sequence (e.g., a second reporter barcode sequence) corresponding to the second antigen or epitope,
wherein the first antigen or epitope of the first cell is bound by one or more antibody molecules or antigen-binding fragments in the sample while the second antigen or epitope of the second cell is not,
wherein the first cell with the bound one or more antibody molecules or antigen-binding fragments is partitioned in a partition, the partition comprising a plurality of barcode nucleic acid molecules which comprise a common partition-specific barcode sequence, and
wherein a barcoded nucleic acid molecule is generated in the partition, and the barcoded nucleic acid molecule comprises (i) the first nucleic acid sequence or a complement thereof and (ii) the partition-specific barcode sequence or a complement thereof.

2. The method of claim 1, wherein the sample comprises a bodily fluid,
wherein the bodily fluid is a blood sample, a serum sample or a plasma sample.

3. (canceled)

4. The method of claim 1, wherein the plurality of antibody molecules or antigen-binding fragments thereof comprises:
(i) two or more antibody molecules or antigen-binding fragments thereof having different: (a) antigen-binding specificities, (b) antigen-binding affinities or (c) antigen-binding specificities and affinities;
(ii) two or more antibody molecules or antigen-binding fragments thereof having the same or substantially the same: (a) antigen-binding specificities (b) antigen-binding affinities or (c) antigen-binding specificities and affinities;
(iii) an antibody molecule or antigen binding fragment thereof that recognizes two or more different antigens or epitopes; or
(iv) one or more monoclonal antibodies.

5-7. (canceled)

8. The method of claim 4(iv), wherein the one or more monoclonal antibodies are coupled to one or more antibody barcode sequences.

9. The method of claim 1, wherein the first, second or first and second antigens or epitopes are heterologous to the first and second cells, respectively.

10. (canceled)

11. The method of claim 1, wherein the first, second, or first and second antigens or epitopes are peptides or proteins.

12-13. (canceled)

14. The method of claim 1, wherein the first and second nucleic acid sequences are different, thereby identifying the first cell and the second cell as expressing different antigens or epitopes.

15. The method of claim 1, wherein the first and second nucleic acid sequences are directly or indirectly coupled to the first and second antigens or epitopes, respectively, wherein the coupled is through chemical conjugation or enzymatic conjugation.

16-17. (canceled)

18. The method of claim 1, wherein the first and second cells are engineered by introducing into the cells an expression system capable of expressing the first and second antigens or epitopes, respectively, in the cells,
wherein the expression system:
(i) is introduced into the genome of the first and second cells,
(ii) comprises coding sequences for the first and second antigens or epitopes, respectively; or
(iii) is introduced into the genome of the first and second cells and comprises coding sequences for the first and second antigens or epitopes, respectively.

19-21. (canceled)

22. The method of claim 18, wherein the first and second cells constitutively express the first and second antigens or epitopes, respectively, from the expression system.

23. The method of claim 18, wherein the expression system comprises first and second antigen barcode sequences or a complements thereof corresponding to the first and second antigen or epitope, respectively, wherein the first and second antigen barcode sequences or complements thereof are distinct from coding sequences for the first and second antigen or epitope, respectively.

24. The method of claim 18, wherein the expression system comprises a sequence or a complement thereof configured to couple to a barcode nucleic acid molecule of the plurality of barcode nucleic acid molecules in the partition.

25. The method of claim 18, wherein a transcript of the expression system is coupled to a barcode nucleic acid molecule of the plurality of barcode nucleic acid molecules in the partition,
wherein the transcript comprises a coding sequence for the first or second antigen or epitope, a first or second antigen barcode sequence or a complement thereof corresponding to the first or second antigen or epitope, respectively, and a sequence or a complement thereof configured to couple the transcript to the barcode nucleic acid molecule in the partition.

26. (canceled)

27. The method of claim 1, wherein during or after the contacting,
cells of the population of cells bound by one or more of the plurality of antibody molecules or antigen-binding fragments thereof in the sample are one or more of: enriched, purified, isolated, sorted, or separated
cells of the population of cells not bound by one or more of the plurality of antibody molecules or antigen-binding fragments thereof in the sample are one or more of: enriched, purified, isolated, sorted, or separated.

28. The method of claim 27, wherein the cells of the population of cells bound by one or more of the plurality of antibody molecules or antigen-binding fragments thereof in the sample are sorted or purified using: (i) a cytometer; (ii) fluorescence-activated cell sorting (FACS); (iii) magnetic-activated cell sorting (MACS); or (iv) buoyancy-activated cell sorting (BACS).

29-31. (canceled)

32. The method of any of claim 1, wherein the barcoded nucleic acid molecule is detected, analyzed, or detected and analyzed by nucleic acid sequencing.

33-34. (canceled)

35. A method for analyzing a sample, comprising:
contacting a sample with a population of cells,
wherein the sample comprises a monoclonal antibody or antigen-binding fragment thereof coupled to an antibody barcode sequence,
wherein the population comprises a cell that is engineered or otherwise modified to express an antigen or epitope and that comprises an antigen barcode sequence corresponding to the antigen or epitope,
wherein the antigen or epitope expressed by the cell is bound by the monoclonal antibody or antigen-binding fragment thereof,
wherein the cell with the bound monoclonal antibody or antigen-binding fragment thereof is partitioned in a partition, the partition comprising a plurality of barcode nucleic acid molecules which comprise a common partition-specific barcode sequence, and
wherein a first barcoded nucleic acid molecule and a second barcoded nucleic acid molecule are generated in the partition, the first barcoded nucleic acid molecule comprises (i) the antibody barcode sequence or a complement thereof and (ii) the partition-specific barcode sequence or a complement thereof, and the second barcoded nucleic acid molecule comprises (i) the antigen barcode sequence or a complement thereof and (ii) the partition-specific barcode sequence or a complement thereof.

36. The method of claim 35, wherein the sample comprises a plurality of monoclonal antibodies or antigen-binding fragments thereof, wherein the plurality of monoclonal antibodies or antigen-binding fragments thereof comprises:
(i) two or more monoclonal antibodies or antigen-binding fragments thereof having different: (a) antigen-binding specificities, (b) antigen-binding specificities or (c) antigen-binding specificities and antigen-binding affinities,
(ii) a monoclonal antibody or antigen-binding fragment thereof that recognizes two or more different antigens or epitopes; or
(iii) both (i) and (ii).

37-38. (canceled)

39. The method of claim 35, wherein the population comprises cells engineered or otherwise modified to express a panel of candidate epitopes comprising the antigen or epitope for the monoclonal antibody or antigen-binding fragment thereof.

40. The method of claim 35, wherein during or after the contacting:
cells of the population of cells bound by monoclonal antibodies or antigen-binding fragments thereof in the sample are one or more of: enriched, purified, isolated, sorted, or separated; or
cells of the population of cells not bound by monoclonal antibodies or antigen-binding fragments thereof in the sample are one or more of: enriched, purified, isolated, sorted, or separated.

41. (canceled)
